(19) United States
(12) Patent Application Publication
(10) Pub. No.: US 2022/0193065 A1
(43) Pub. Date: Jun. 23, 2022

(54) COMPOSITIONS AND METHODS TO TREAT NON-ALCOHOLIC FATTY LIVER DISEASES (NAFLD)

(71) Applicant: Coherus Biosciences, Inc., Redwood City, CA (US)

(72) Inventors: Christos Mantzoros, Watertown, MA (US); Glenn D. Rosen, Los Altos, CA (US)

(21) Appl. No.: 17/600,172

(22) PCT Filed: Dec. 27, 2019
(86) PCT No.: PCT/US2019/068704

§ 371 Date: Sep. 30, 2021

Related U.S. Application Data
(60) Provisional application No. 62/829,075, filed on Apr. 4, 2019.

(51) Int. Cl. A61K 31/47; A61P 1/16; A61K 31/355

(52) U.S. Cl. CPC A61K 31/47; A61K 31/355; A61P 1/16

(57) ABSTRACT

Provided herein are methods and combination therapies useful for the treatment of non-alcoholic fatty liver diseases (NAFLD). In particular, provided herein are methods and combination therapies for treating NAFLD by administering a combination therapy comprising (a) the compound of Formula (I), or a pharmaceutically acceptable salt thereof, and (b) an antioxidant, or a pharmaceutically acceptable salt thereof. Also provided are pharmaceutical compositions and pharmaceutical combinations comprising the compound of Formula (I), or a pharmaceutically acceptable salt thereof, and an antioxidant, or a pharmaceutically acceptable salt thereof.

### CROSS REFERENCE TO RELATED APPLICATIONS

This application claims priority to U.S. Provisional Application Ser. No. 62/829,075, filed on Apr. 4, 2019, which is herein incorporated by reference in its entirety.

### TECHNICAL FIELD

The present disclosure relates to methods and combination therapies useful for the treatment of non-alcoholic fatty liver diseases (NAFLD). In particular, this disclosure relates to methods and combination therapies for treating NAFLD by administering a combination therapy comprising a PPARγ inhibitor that is the compound of Formula (I), or a pharmaceutically acceptable salt thereof, and an antioxidant (e.g., vitamins, minerals, polyphenols, anthocyanins, carotenoids, or glucosinolates), or a pharmaceutically acceptable salt thereof.

### BACKGROUND

Non-alcoholic fatty liver disease (NAFLD) is characterized by the presence of hepatic fat accumulation in the absence of secondary causes of hepatic steatosis including excessive alcohol consumption, other known liver diseases, or long-term use of a steatogenic medication (Perumpail et al., World J Gastroenterol. 2017, 23(47):8263-8438 and Chalasani et al., Hepatology. 2018, 67(1):328-357). NAFLD encompasses two categories: simple non-alcoholic fatty liver (NAFL) and non-alcoholic steatohepatitis (NASH). Typically, NAFL has a more indolent course of progression whereas NASH is a more severe form associated with inflammation that may progress more rapidly to end-stage liver disease. NAFL and/or NASH may also include scarring of the liver known as liver fibrosis or in a more severe form, liver cirrhosis. Scarring of the liver reduces liver function up to and including liver failure.

NAFLD is currently the most common liver disease in the world (Perumpail et al., World J Gastroenterol. 2017, 23(47):8263-8438) with approximately one-fourth of the adult population suffering from NAFLD worldwide (Sumida, et al., J Gastroenterol. 2018, 53:362-376). There are many risk factors associated with NAFLD including hypertension, obesity, diabetes, and hyperipidemia with a particularly close association with type II diabetes mellitus and NAFLD (Vernon et al., Aliment Pharmacol Ther. 2011, 34:274-285).

Lifestyle interventions including dietary caloric restriction and exercise are the most effective methods of prevention and treatment for NAFLD (Sumida, et al., J Gastroenterol. 2018, 53:362-376). However, these can be difficult treatments to follow. Thus, there is a need for pharmaceuticals to treat NAFLD. Current pharmaceutical treatments that have been proposed or tested in prior trials, although are not yet approved for NAFLD include vitamin E, ω3 fatty acid, statin, metformin, orlistat, thiazolidinediones (“TZDs”), urodeoxycholic acid, pioglitazone, and pentoxifilline (Sumida, et al., J Gastroenterol. 2018, 53:362-376). However, there is currently no approved pharmacotherapy for NAFLD.

While some treatments have shown early promise in clinical trials, others have failed to shows efficacy as a monotherapy. For example, selonsertib—an apoptosis signal-regulating kinase 1 inhibitor—failed to meet the primary endpoint in the STELLAR-4 phase 3 clinical trial. While a single treatment may not be efficacious in treating NAFLD, a combination of therapies may be efficacious. There is a need to identify combinations of therapeutic agents that will efficacious in treating NAFLD.

There is increasing evidence suggesting that oxidative stress is a key driving force in the progression of NAFLD. Oxidative stress can cause direct damage to lipid, protein, and DNA molecules and trigger the inflammatory and fibrogenesis signaling pathways. Indeed, elevated levels of oxidative stress are well documented in NAFLD patients. Liu, et al., Curr. Drug Tar., 2015, 16(12): 1301-14. The elevated levels of oxidative stress (e.g., over production of reactive oxygen species) deplete endogenous antioxidant molecules such as glutathione and inhibit the activities of antioxidant enzymes such as superoxide dismutase.

### SUMMARY

Provided herein in some embodiments is a method of treating non-alcoholic fatty liver disease (NAFLD) in a subject in need thereof comprising administering to the subject
(a) the compound of Formula (I),

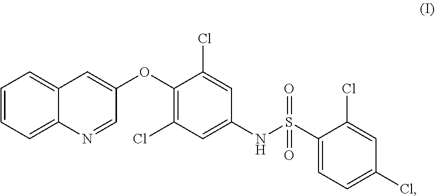 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) an antioxidant, or a pharmaceutically acceptable salt thereof, wherein the amounts of (a) and (b) together are effective in treating NAFLD.

Provided herein in some embodiments is a method of treating a subject, the method comprising:

selecting a subject having non-alcoholic fatty liver disease (NAFLD); and

administering
(a) the compound of Formula (I),

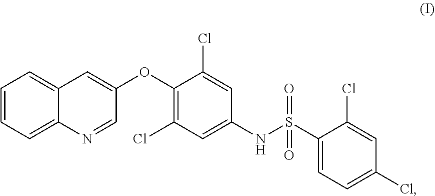 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) an antioxidant, or a pharmaceutically acceptable salt thereof, to the selected subject, wherein the amounts of (a) and (b) together are effective in treating NAFLD.

Provided herein in some embodiments is a method of treating a subject, the method comprising:

identifying a subject having non-alcoholic fatty liver disease (NAFLD); and

administering
(a) the compound of Formula (I),

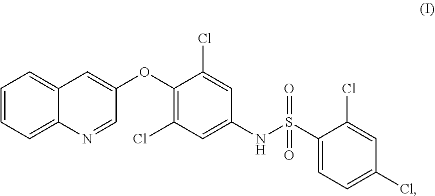 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) an antioxidant, or a pharmaceutically acceptable salt thereof, to the selected subject, wherein the amounts of (a) and (b) together are effective in treating NAFLD.

Provided herein in some embodiments is a method of treating non-alcoholic fatty liver disease (NAFLD) in a subject in need thereof comprising administering to the subject
(a) a therapeutically effective amount of the compound of Formula (I),

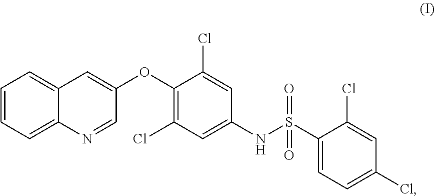 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) a therapeutically effective amount of an antioxidant, or a pharmaceutically acceptable salt thereof.

Provided herein in some embodiments is a method of treating a subject, the method comprising:
selecting a subject having non-alcoholic fatty liver disease (NAFLD); and
administering
(a) a therapeutically effective amount of the compound of Formula (I),

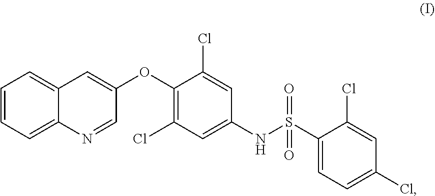 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) a therapeutically effective amount of an antioxidant, or a pharmaceutically acceptable salt thereof, to the selected subject.

Provided herein in some embodiments is a method of treating fibrosis in a subject in need thereof comprising administering to the subject
(a) the compound of Formula (I),

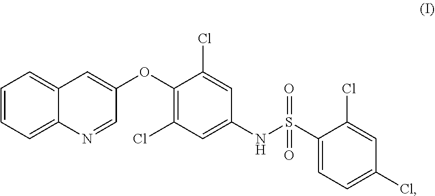 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) an antioxidant, or a pharmaceutically acceptable salt thereof, wherein the amounts of (a) and (b) together are effective in treating fibrosis.

Provided herein in some embodiments is a method of treating fibrosis in a subject in need thereof comprising administering to the subject
(a) a therapeutically effective amount of the compound of Formula (I),

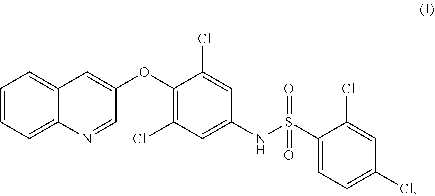 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) a therapeutically effective amount of an antioxidant, or a pharmaceutically acceptable salt thereof.

Provided herein in some embodiments is a method of treating hepatic steatosis in a subject in need thereof comprising administering to the subject
(a) the compound of Formula (I),

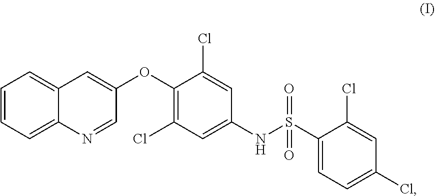 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) an antioxidant, or a pharmaceutically acceptable salt thereof, wherein the amounts of (a) and (b) together are effective in treating hepatic steatosis.

Provided herein in some embodiments is a method of treating hepatic steatosis in a subject in need thereof comprising administering to the subject
(a) a therapeutically effective amount of the compound of Formula (I),

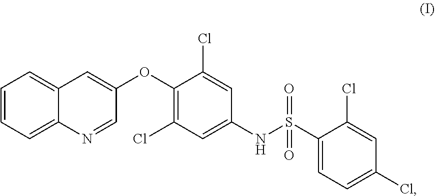 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) a therapeutically effective amount of an antioxidant, or a pharmaceutically acceptable salt thereof.

In some more particular embodiments, (a) and (b) are administered concurrently.

In some more particular embodiments, (a) and (b) are administered sequentially in either order.

Provided herein in some embodiments is a pharmaceutical composition comprising
(a) the compound of Formula (I),

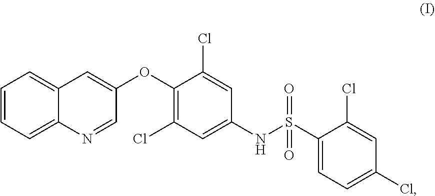 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(b) an antioxidant, or a pharmaceutically acceptable salt thereof, and one or more pharmaceutical excipients.

In some embodiments, the amounts of (a) and (b) together are effective in treating NAFLD.

In some embodiments, the amount of (a) is a therapeutically effective amount and the amount of (b) is a therapeutically effective amount.

Provided herein in some embodiments is a pharmaceutical combination comprising
(a) the compound of Formula (I),

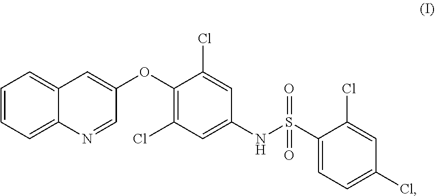 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(b) an antioxidant, or a pharmaceutically acceptable salt thereof, and one or more pharmaceutical excipients, for concurrent or sequential administration during a period of time for use in the treatment of non-alcoholic fatty liver disease (NAFLD).

In some embodiments, the amounts of (a) and (b) together are effective in treating NAFLD.

In some embodiments of the pharmaceutical compositions provided herein, the pharmaceutical compositions comprise at least one pharmaceutically acceptable carrier.

In some more particular embodiments, a method as provided herein comprises administering a pharmaceutical composition as provided herein to a subject twice a day, daily, every other day, three times a week, twice a week, weekly, every other week, twice a month, or monthly.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE FIGURES

FIG. 1 provides an outline for a study to assess the effects of treatment with CHS-131 (Compound of Formula (I)), alone and in combination with other therapeutic agents, to treat NASH, as described in Example 2.

### DETAILED DESCRIPTION

### Definitions

Reference to the term “about” has its usual meaning in the context of pharmaceutical compositions to allow for reasonable variations in amounts that can achieve the same effect and also refers herein to a value of plus or minus 10% of the provided value. For example, “about 20” means or includes amounts from 18 up to and including 22.

The term “administration” or “administering” refers to a method of giving a dosage of a compound or pharmaceutical composition to a vertebrate or invertebrate, including a mammal, a bird, a fish, or an amphibian. The preferred method of administration can vary depending on various factors, e.g., the components of the pharmaceutical composition, the site of the disease, and the severity of the disease.

The term “CHS-131” as used herein refers to a compound of Formula (I):

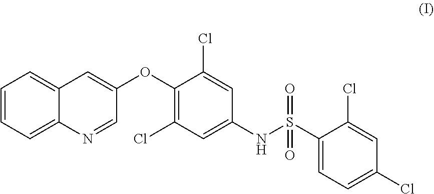 [SMILES: O=S(=O)(Nc1cc(Cl)c(Oc2cnc3ccccc3c2)c(Cl)c1)c1ccc(Cl)cc1Cl]

or a pharmaceutically acceptable salt thereof.

The compound of Formula (I) is a selective peroxisome proliferator-activated receptor (PPAR) γ modulator. The compound of Formula (I) is disclosed in, for example, U.S. Pat. Nos. 7,041,691; 6,200,995; 6,583,157; 6,653,332; and U.S. Publication Application No. 2016/0260398, the contents of each of which are incorporated by reference herein in their entireties.

The compound of Formula (I) can be prepared, for example, by the methods described in U.S. Pat. Nos. 6,583,157 or 6,200,995, each of which is incorporated by reference in its entirety herein. In some embodiments, different salts, e.g., besylate, tosylate HCl, or HBr salts, and/or polymorphs of the compound of Formula (I) are used within the methods and compositions described herein. Salts and polymorphs of the compound of Formula (I), such as those provided herein, can be prepared according to the methods described in U.S. Pat. Nos. 6,583,157 and 7,223,761, the contents of each of which are incorporated by reference in their entireties.

The term “antioxidant” as used herein refers to a compound that inhibits oxidation, for example, oxidation caused by free radicals, and that is suitable for consumption by a mammal.

Examples of antioxidants include, but are not limited to vitamins, minerals, polyphenols, anthocyanins, carotenoids, and glucosinolates. For example,

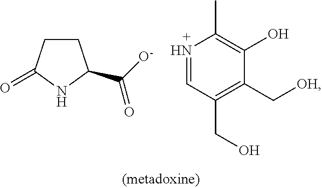

vitamin A, vitamin B1, vitamin B2, vitamin B3, vitamin B5, vitamin B6, vitamin B12, folic acid, vitamin C, vitamin D, vitamin E, selenium, N-acteylcysteine, pyrrolidinedithiocarbamate,

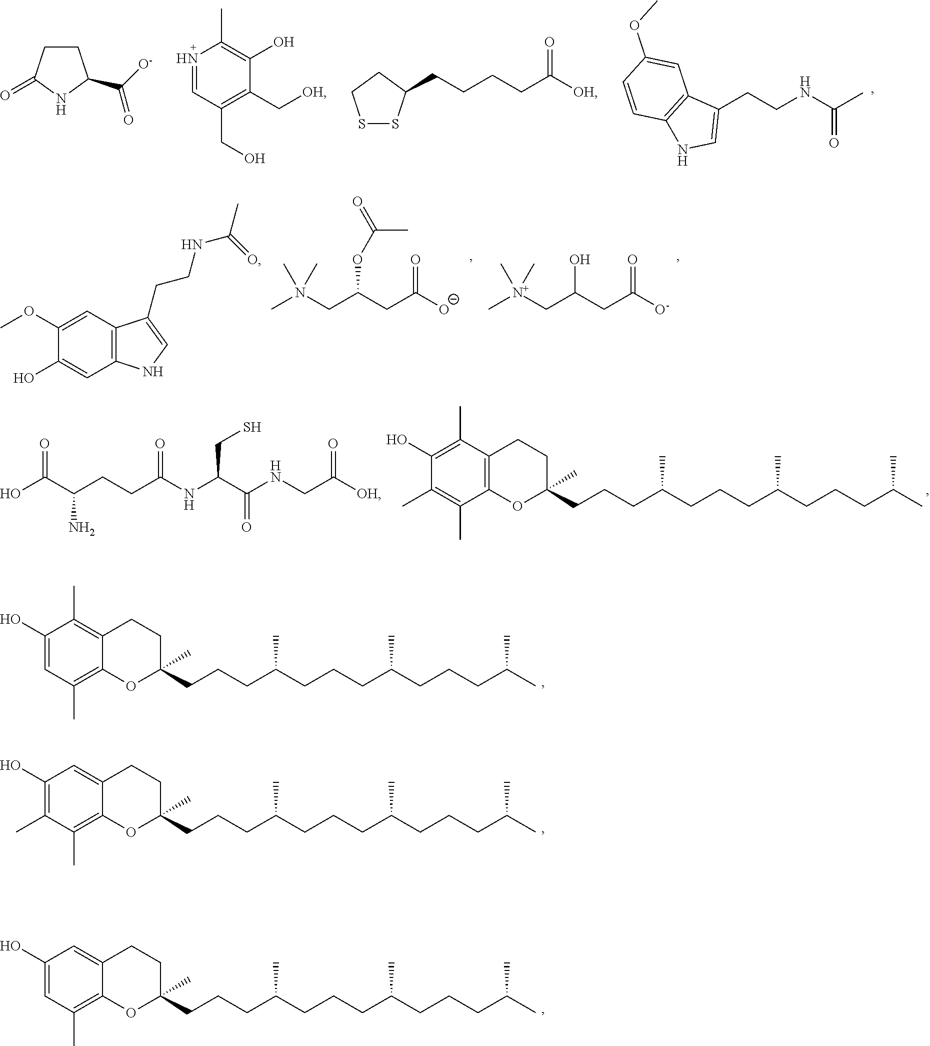

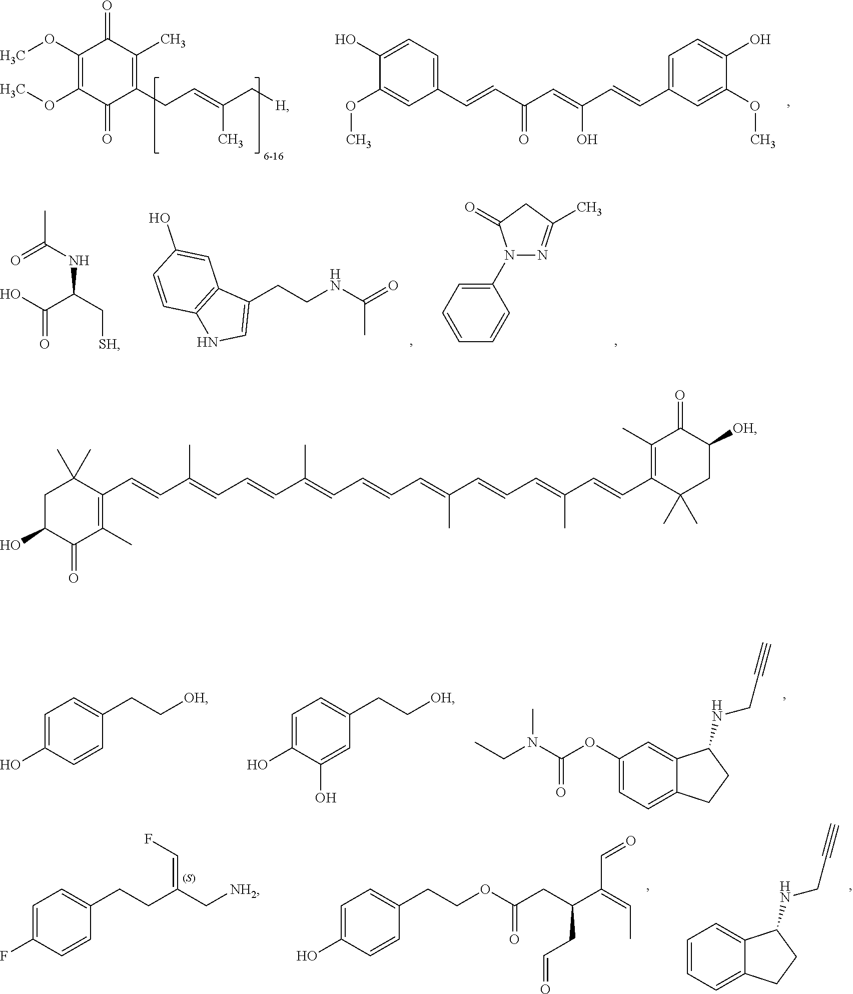

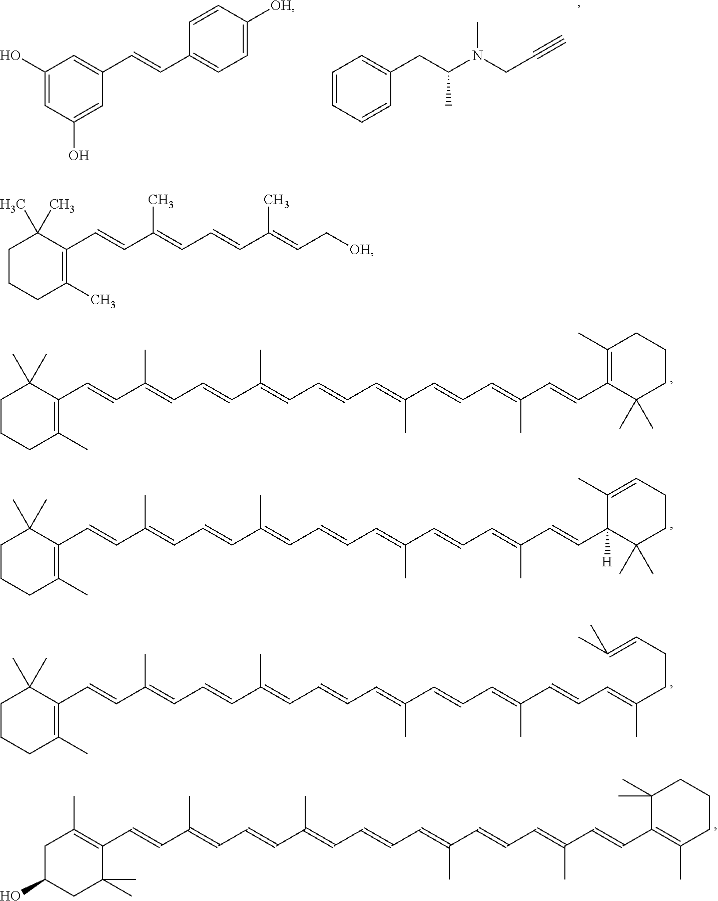

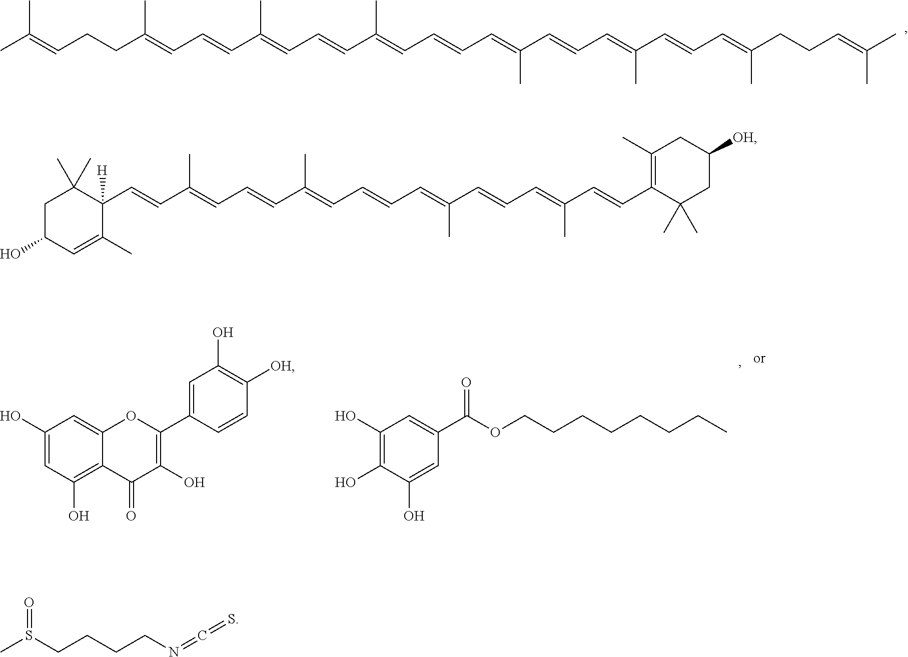

In some embodiments described herein, the compound of Formula I is a free base. In other embodiments, the compound of Formula I is a pharmaceutically acceptable salt, for example a hydrochloride salt. In some embodiments, the antioxidant is a free base. In other embodiments, the antioxidant is a pharmaceutically acceptable salt, for example, a hydrochloride salt.

By “effective dosage” or “therapeutically effective amount” or “pharmaceutically effective amount” of a compound as provided herein is an amount that is sufficient to achieve the desired therapeutic effect and can vary according to the nature and severity of the disease condition, and the potency of the compound. In some embodiments, the therapeutic effect is determined from one or more parameters selected from the NAFLD Activity Score (NAS), hepatic steatosis, hepatic inflammation, biomarkers indicative of liver damage, and liver fibrosis and/or liver cirrhosis. For example, a therapeutic effect can include one or more of a decrease in symptoms, a decrease in the NAS, a reduction in the amount of hepatic steatosis, a decrease in hepatic inflammation, a decrease in the level of biomarkers indicative of liver damage, and a reduction in liver fibrosis and/or liver cirrhosis, a lack of further progression of liver fibrosis and/or liver cirrhosis, or a slowing of the progression of liver fibrosis and/or liver cirrhosis following administration of a compound or compounds as described herein.

A “therapeutic effect,” as used herein, refers to the relief, to some extent, of one or more of the symptoms of the disease, and can include curing a disease. “Curing” means that the symptoms of active disease are eliminated. However, certain long-term or permanent effects of the disease can exist even after a cure is obtained (such as, e.g., extensive tissue damage). In some embodiments, a therapeutically effective amount of a compound as provided herein refers to an amount of the compound that is effective as a monotherapy.

The term “synergy” or “synergistic” is used herein to mean that the effect of the combination of the two therapeutic agents of the combination therapy is greater than the sum of the effect of each agent when administered alone. A “synergistic amount” or “synergistically effective amount” is an amount of the combination of the two combination partners that results in a synergistic effect, as “synergistic” is defined herein. Determining a synergistic interaction between two combination partners, the optimum range for the effect and absolute dose ranges of each component for the effect may be definitively measured by administration of the combination partners over different w/w (weight per weight) ratio ranges and doses to patients in need of treatment. However, the observation of synergy in in vitro models or in vivo models can be predictive of the effect in humans and other species and in vitro models or in vivo models exist, as described herein, to measure a synergistic effect and the results of such studies can also be used to predict effective dose and plasma concentration ratio ranges and the absolute doses and plasma concentrations required in humans and other species by the application of pharmacokinetic/pharmacodynamic methods. Exemplary synergistic effects includes, but are not limited to, enhanced therapeutic efficacy, decreased dosage at equal or increased level of efficacy, reduced or delayed development of drug resistance, and simultaneous enhancement or equal therapeutic actions (e.g., the same therapeutic effect as at least one of the therapeutic agents) and reduction of unwanted drug effects (e.g., side effects and adverse events) of at least one of the therapeutic agents.

For example, a synergistic ratio of two therapeutic agents can be identified by determining a synergistic effect in, for example, an art-accepted in vivo model (e.g., an animal model) of NAFLD (e.g., the diet induced obese (DIO)-NASH mouse model or any of the models described in Van Herck et al. Nutrients. 2017 October; 9(10): 1072, and Kristiansen et al. World J Hepatol. 2016; 8(16):673-84, which are incorporated by reference herein in their entirety). In one embodiment of a DIO-NASH model, the mouse model is induced by feeding male C57BL/6JRj mice a high fat diet containing 40% fat with trans-fat, 20% fructose and 2% cholesterol (AMLN diet or D09100301, Research Diets Inc., USA). In another embodiment, the model is a male Lep^{ob}/Lep^{ob} (ob/ob) mouse model.

The term “preventing” as used herein means the prevention of the onset, recurrence or spread, in whole or in part, of the disease or condition as described herein, or a symptom thereof.

As used herein, the terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, “subject” or “patient” refers to any subject, particularly a mammalian subject, for whom diagnosis, prognosis, or therapy is desired, for example, a human.

The terms “treatment regimen” and “dosing regimen” are used interchangeably to refer to the dose and timing of administration of each therapeutic agent in a combination of the invention.

The term “pharmaceutical combination,” as used herein, refers to a pharmaceutical treatment resulting from the mixing or combining of more than one active ingredient and includes both fixed and non-fixed combinations of the active ingredients.

The term “combination therapy,” as used herein refers to a dosing regimen of two different therapeutically active agents (i.e., the components or combination partners of the combination) (e.g., the compound of Formula (I), or a pharmaceutically acceptable salt thereof, and an antioxidant), wherein the therapeutically active agents are administered together or separately in a manner prescribed by a medical care taker or according to a regulatory agency as defined herein. In one embodiment, a combination therapy comprises a combination of the compound of Formula (I), or a pharmaceutically acceptable salt thereof, and antioxidant (e.g., vitamins, minerals, polyphenols, anthocyanins, carotenoids, or glucosinolates), or a pharmaceutically acceptable salt thereof. In one embodiment, a combination therapy consists essentially of a combination of (a) the compound of Formula (I), or a pharmaceutically acceptable salt thereof, and (b) an antioxidant, or a pharmaceutically acceptable salt thereof (e.g., vitamins, minerals, polyphenols, anthocyanins, carotenoids, or glucosinolates).

The term “fixed combination” means that the compound of Formula (I), or a pharmaceutically acceptable salt thereof, and an additional therapeutic agent (e.g., an antioxidant, or a pharmaceutically acceptable salt thereof), are each administered to a subject simultaneously in the form of a single composition or dosage.

The term “non-fixed combination” means that the compound of Formula (I), or a pharmaceutically acceptable salt thereof, and an additional therapeutic agent (e.g., an antioxidant, or a pharmaceutically acceptable salt thereof) are formulated as separate compositions or dosages such that they may be administered to a subject in need thereof concurrently or sequentially with variable intervening time limits, wherein such administration provides effective levels of the two or more compounds in the body of the subject. These also apply to cocktail therapies, e.g., the administration of three or more active ingredients.

As can be appreciated in the art, a combination therapy can be administered to a patient for a period of time. In some embodiments, the period of time occurs following the administration of a different therapeutic treatment/agent or a different combination of therapeutic treatments/agents to the patient. In some embodiments, the period of time occurs before the administration of a different therapeutic treatment/agent or a different combination of therapeutic treatments/agents to the subject.

A suitable period of time can be determined by one skilled in the art (e.g., a physician). As can be appreciated in the art, a suitable period of time can be determined by one skilled in the art based on one or more of: the stage of disease in the patient, the mass and sex of the patient, clinical trial guidelines (e.g., those on the fda.gov website), and information on the approved drug label. In some embodiments, a suitable period of time can be from 1 week to 2 years, for example, 1 week, 2, weeks, 4 weeks, 6 weeks, 8 weeks, 12 weeks, 16 weeks, 6 months, 9 months, 12 months, 18 months, or 2 years, or any value in between. In other embodiments, a suitable period of time can be from 1 month to 10 years, for example, 1 month, 6 months, 1 year, 18 months, 2 years, 3 years, 4 years, 5 years, 6 years, 7 years, 8 years, 9 years, or 10 years, or any value in between

The phrases “prior to a period of time” or “before a period of time” refer to (1) the completion of administration of treatment to the subject before the first administration of a therapeutic agent during the period of time, and/or (2) the administration of one or more therapeutic agents to the subject before a first administration of a therapeutic agent in the combination therapy described herein during the period of time, such that the one or more therapeutic agents are present in subtherapeutic and/or undetectable levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in subtherapeutic levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in undetectable levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in subtherapeutic and/or undetectable levels in the subject at the time the first administration of a therapeutic agent in the combination therapy is performed during the period of time.

In some embodiments, a combination of (a) the compound of Formula (I), or a pharmaceutically acceptable salt thereof, and (b) an antioxidant, or a pharmaceutically acceptable salt thereof, produces a synergistic effect; for example, any of the beneficial or desired results including clinical results as described herein, for example slowing the symptomatic progression of NAFLD, or symptoms thereof, which is greater than the sum of effect observed when the compound of Formula (I), or a pharmaceutically acceptable salt thereof, and the antioxidant, or a pharmaceutically acceptable salt thereof are each administered alone.

In some embodiments, a combination of (a) the compound of Formula (I), or a pharmaceutically acceptable salt thereof, and (b) an antioxidant, or a pharmaceutically acceptable salt thereof, produces a synergistic effect; for example, any of the beneficial or desired results including clinical results as described herein, for example slowing the symptomatic progression of NAFLD, or symptoms thereof, which is greater than the sum of effect observed when the same amount of the compound of Formula (I), or a pharmaceutically acceptable salt thereof, and the same amount of the antioxidant, or a pharmaceutically acceptable salt thereof as in the combination are each administered alone.

In some more particular embodiments a combination of (a) the compound of Formula (I), or a pharmaceutically acceptable salt thereof, and (b) an antioxidant, or a pharmaceutically acceptable salt thereof, produces a synergistic effect, for example, a therapeutic effect using a smaller dose of either or both of (a) and (b), compared to the amount used in monotherapy. In some embodiments, the dose of (a), administered in combination with (b) may be about 0.5% to about 90% of the dose of (a) administered as a monotherapy to produce the same therapeutic effect, e.g., any of the beneficial or desired results including clinical results as described herein, for example slowing the symptomatic progression of NAFLD, or symptoms thereof. In some embodiments, the dose of (a) administered in combination with (b), may be about 0.5% to 30%, about 30% to about 60%, about 60% to about 90%, such as about 0.5%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% of the dose of (a) administered as a monotherapy. As another example, the dose of the (b) administered in combination with (a) may be about 0.5% to about 90% of the dose of (b) administered as a monotherapy to produce the same therapeutic effect, e.g., any of the beneficial or desired results including clinical results as described herein, for example slowing the symptomatic progression of NAFLD, or symptoms thereof.

In some embodiments of the methods or combinations herein, a subject may be administered an amount of a compound that produces a therapeutic effect in the absence of another compound of the combinations disclosed herein. In particular embodiments of the methods or combinations herein, a subject may be administered two compounds which together produce a therapeutic effect. For example, two compounds when dosed together may have an additive or synergistic effect, such that the dose of each individual compound may independently be an effective amount, or may be a sub-therapeutic amount, but together the total amount of the combination of compounds provides a therapeutically effective amount.

In some embodiments, the amounts of the two or more compounds as provided herein together are effective in treating NAFLD (e.g., the amounts of the compound of Formula (I) and an antioxidant together are effective in treating NAFLD). For example, wherein the amounts of (a) the compound of Formula (I), or a pharmaceutically acceptable salt thereof, (b) an antioxidant, or a pharmaceutically acceptable salt thereof, together are effective in treating NAFLD, the therapeutic effect of the combination of (a) and (b) is 10%-100% greater than, such as 10%-50%, 20%-60%, 30%-70%, 40%-80%, 50%-90%, or 60%-100%, greater than, such as 10%, 20%, 30%, 40%, 50%, 60%, 70%, 80%, or 90% greater than, the therapeutic effect of the compound of Formula (I), or a pharmaceutically acceptable salt, prodrug, or isomer thereof alone. In some embodiments, wherein the amounts of (a) and (b) are effective in treating NAFLD, the therapeutic effect of the combination of (a) and (b) is 10%-100% greater than, such as 10%-50%, 20%-60%, 30%-70%, 40%-80%, 50%-90%, or 60%-100%, greater than, such as 10%, 20%, 30%, 40%, 50%, 60%, 70%, 80%, or 90% greater than, the therapeutic effect of (a) alone, or (b) alone (i.e., administered as a monotherapy).

In some more particular embodiments, a combination of (a) the compound of Formula (I), or a pharmaceutically acceptable salt thereof, and (b) an antioxidant, or a pharmaceutically acceptable salt thereof, produces a synergistic effect: the desired therapeutic effect and a reduction in an unwanted drug effect, side effect, or adverse event.

In some more particular embodiments, a combination of (a) the compound of Formula (I), or a pharmaceutically acceptable salt thereof, and (b) an antioxidant, or a pharmaceutically acceptable salt thereof, produces a synergistic effect: the desired therapeutic effect and a reduction in an unwanted drug effect, side effect, or adverse event.

In some embodiments, the desired therapeutic effect is the same therapeutic effect observed in monotherapy of the compound of Formula (I), or a pharmaceutically acceptable salt thereof, an antioxidant, or a pharmaceutically acceptable salt thereof, e.g., any of the beneficial or desired results including clinical results as described herein, for example slowing the symptomatic progression of NAFLD, or symptoms thereof.

In some embodiments, an unwanted drug effect, side effect, or adverse event is associated with or observed in monotherapy of the compound of Formula (I), or a pharmaceutically acceptable salt thereof, or an antioxidant, or a pharmaceutically acceptable salt thereof. For example, an unwanted drug effect, side effect, or adverse event includes, but is not limited to edema, weight gain, hypertension, cardiovascular disease, cardiovascular events (e.g., cardiovascular death, nonfatal myocardial infarction and nonfatal stroke), and combinations thereof.

### Methods and Combination Therapies

The present disclosure relates to methods and combination therapies for treating non-alcoholic fatty liver disease (NAFLD) in a subject in need thereof by administering (a) the compound of Formula (I):

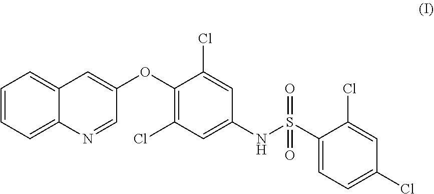 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and (b) an antioxidant, or a pharmaceutically acceptable salt thereof.

NAFLD is characterized by hepatic steatosis with no secondary causes of hepatic steatosis including excessive alcohol consumption, other known liver diseases, or long-term use of a steatogenic medication (Chalasani et al., Hepatology. 2018, 67(1):328-357, which is hereby incorporated by reference in its entirety). NAFLD can be categorized into non-alcoholic fatty liver (NAFL) and non-alcoholic steatohepatitis (NASH). According to Chalasani et al., NAFL is defined as the presence of ≥5% hepatic steatosis without evidence of hepatocellular injury in the form of hepatocyte ballooning. NASH is defined as the presence of ≥5% hepatic steatosis and inflammation with hepatocyte injury (e.g., ballooning), with or without any liver fibrosis. Additionally, NASH is commonly associated with hepatic inflammation and liver fibrosis, which can progress to cirrhosis, end-stage liver disease, and hepatocellular carcinoma. However, liver fibrosis is not always present in NASH, but the severity of fibrosis can be linked to long-term outcomes.

There are many approaches used to assess and evaluate whether a subject has NAFLD and if so, the severity of the disease including differentiating whether the NAFLD is NAFL or NASH. For example, these approaches include determining one or more of hepatic steatosis (e.g., accumulation of fat in the liver); the NAFLD Activity Score (NAS); hepatic inflammation; biomarkers indicative of one or more of liver damage, hepatic inflammation, liver fibrosis, and/or liver cirrhosis (e.g., serum markers and panels); and liver fibrosis and/or cirrhosis. Further examples of physiological indicators of NAFLD can include liver morphology, liver stiffness, and the size or weight of the subject's liver. In some embodiments, NAFLD in the subject is evidenced by an accumulation of hepatic fat and detection of a biomarker indicative of liver damage. For example, elevated serum ferritin and low titers of serum autoantibodies can be common features of NAFLD. In some embodiments, methods to assess NAFLD include magnetic resonance imaging, either by spectroscopy or by proton density fat fraction (MRI-PDFF) to quantify steatosis, transient elastography (FIBROSCAN®), hepatic venous pressure gradient (HPVG), hepatic stiffness measurement with MRE for diagnosing significant liver fibrosis and/or cirrhosis, and assessing histological features of liver biopsy. In some embodiments, magnetic resonance imaging is used to detect one or more of steatohepatitis (NASH-MRI), liver fibrosis (Fibro-MRI), and steatosis see, for example, U.S. Application Publication Nos. 2016/146715 and 2005/0215882, each of which are incorporated herein by reference in their entireties. In some embodiments, treatment of NAFLD comprises one or more of a decrease in symptoms; a reduction in the amount of hepatic steatosis; a decrease in the NAS; a decrease in hepatic inflammation; a decrease in the level of biomarkers indicative of one or more of liver damage, inflammation, liver fibrosis, and/or liver cirrhosis; and a reduction in fibrosis and/or cirrhosis, a lack of further progression of fibrosis and/or cirrhosis, or a slowing of the progression of fibrosis and/or cirrhosis.

In some embodiments, treatment of NAFLD comprises a decrease of one or more symptoms associated with NAFLD in the subject. Exemplary symptoms can include one or more of an enlarged liver, fatigue, pain in the upper right abdomen, abdominal swelling, enlarged blood vessels just beneath the skin's surface, enlarged breasts in men, enlarged spleen, red palms, jaundice, and pruritus. In some embodiments, the subject is asymptomatic. In some embodiments, the total body weight of the subject does not increase. In some embodiments, the total body weight of the subject decreases. In some embodiments, the body mass index (BMI) of the subject does not increase. In some embodiments, the body mass index (BMI) of the subject decreases. In some embodiments, the waist and hip (WTH) ratio of the subject does not increase. In some embodiments, the waist and hip (WTH) ratio of the subject decreases.

In some embodiments, hepatic steatosis is determined by one or more methods selected from the group consisting of ultrasonography, computed tomography (CT), magnetic resonance imaging, magnetic resonance spectroscopy (MRS), magnetic resonance elastography (MRE), transient elastography (TE) (e.g., FIBROSCAN®), measurement of liver size or weight, or by liver biopsy (see, e.g., Di Lascio et al., Ultrasound Med Biol. 2018 August; 44(8):1585-1596; Lv et al., J Clin Transl Hepatol. 2018 Jun. 28; 6(2): 217-221; Reeder, et al., J Magn Reson Imaging. 2011 October; 34(4): 848-855; and de Ledinghen V, et al., J Gastroenterol Hepatol. 2016 April; 31(4):848-55, each of which are incorporated herein by reference in their entireties). A subject diagnosed with NAFLD can have more than about 5% hepatic steatosis, for example, about 5% to about 25%, about 25% to about 45%, about 45% to about 65%, or greater than about 65% hepatic steatosis. In some embodiments, a subject with about 5% to about 33% hepatic steatosis has stage 1 hepatic steatosis, a subject with about 33% to about 66% hepatic steatosis has stage 2 hepatic steatosis, and a subject with greater than about 66% hepatic steatosis has stage 3 hepatic steatosis. In some embodiments, treatment of NAFLD can be assessed by measuring hepatic steatosis. In some embodiments, treatment of NAFLD comprises a reduction in hepatic steatosis following administration of one or more compounds described herein.

In some embodiments, the amount of hepatic steatosis is determined prior to administration of the combination of (a) the compound of Formula (I), or a pharmaceutically acceptable salt thereof, and (b) an antioxidant, or a pharmaceutically acceptable salt thereof. In some embodiments, the amount of hepatic steatosis is determined during the period of time or after the period of time of administration of the combination of (a) and (b). In some embodiments, a reduction in the amount of hepatic steatosis during the period of time or after the period of time of administration of the combination of (a) and (b) compared to prior to administration of the combination of (a) and (b) indicates treatment of NAFLD. For example, a reduction in the amount of hepatic steatosis by about 1% to about 50%, about 25% to about 75%, or about 50% to about 100% indicates treatment of NAFLD. In some embodiments, a reduction in the amount of hepatic steatosis by about 5%, bout 10%, about 15%, about 20%, about 25%, about 30%, about 35%, about 40%, about 45%, about 50%, about 55%, about 60%, about 65%, about 70%, about 75%, about 80%, about 85%, about 90%, or about 95% indicates treatment of NAFLD.

In some embodiments, the severity of NALFD can be assessed using the NAS. In some embodiments, treatment of NAFLD can be assessed using the NAS. In some embodiments, treatment of NAFLD comprises a reduction in the NAS following administration of one or more compounds described herein. In some embodiments, the NAS can be determined as described in Kleiner et al., Hepatology. 2005, 41(6):1313-1321, which is hereby incorporated by reference in its entirety. See, for example, Table 1 for a simplified NAS scheme adapted from Kleiner.

TABLE 1
Example of the NAFLD Activity Score
(NAS) with Fibrosis Stage

| Feature | Degree | Score |
|---|---|---|
| Steatosis | <5% | 0 |
| | 5-33% | 1 |
| | >33-66% | 2 |
| | >66% | 3 |
| Lobular | No foci | 0 |
| inflammation | <2 foci/200x | 1 |
| | 2-4 foci/200x | 2 |
| | >4 foci/200x | 3 |
| Ballooning | None | 0 |
| degeneration | Few | 1 |
| | Many cells/Prominent ballooning | 2 |
| Fibrosis | None | 0 |
| | Perisinusoidal or periportal | 1 |
| | Perisinusoidal & portal/periportal | 2 |
| | Bridging fibrosis | 3 |
| | Cirrhosis | 4 |

In some embodiments, the NAS is determined non-invasively, for example, as described in U.S. Application Publication No. 2018/0140219, which is incorporated by reference herein in its entirety. In some embodiments, the NAS is determined for a sample from the subject prior to administration of the combination of (a) the compound of Formula (I), or a pharmaceutically acceptable salt thereof, and (b) an antioxidant, or a pharmaceutically acceptable salt thereof. In some embodiments, the NAS is determined during the period of time or after the period of time of administration of the combination of (a) and (b). In some embodiments, a lower NAS score during the period of time or after the period of time of administration of the combination of (a) and (b) compared to prior to administration of the combination of (a) and (b) indicates treatment of NAFLD. For example, a decrease in the NAS by 1, by 2, by 3, by 4, by 5, by 6, or by 7 indicates treatment of NAFLD. In some embodiments, the NAS following administration of the combination of (a) and (b) is 7 or less. In some embodiments, the NAS during the period of time of administration of the combination of (a) and (b) is 5 or less, 4 or less, 3 or less, or 2 or less. In some embodiments, the NAS during the period of time of administration of the combination of (a) and (b) is 7 or less. In some embodiments, the NAS during the period of time of administration of the combination of (a) and (b) is 5 or less, 4 or less, 3 or less, or 2 or less. In some embodiments, the NAS after the period of time of administration of the combination of (a) and (b) is 7 or less. In some embodiments, the NAS after the period of time of administration of the combination of (a) and (b) is 5 or less, 4 or less, 3 or less, or 2 or less.

In some embodiments, the presence of hepatic inflammation is determined by one or more methods selected from the group consisting of biomarkers indicative of hepatic inflammation and a liver biopsy sample(s) from the subject. In some embodiments, the severity of hepatic inflammation is determined from a liver biopsy sample(s) from the subject. For example, hepatic inflammation in a liver biopsy sample can be assessed as described in Kleiner et al., Hepatology. 2005, 41(6):1313-1321 and Brunt et al., Am J Gastroenterol 1999, 94:2467-2474, each of which are hereby incorporated by reference in their entireties. In some embodiments, the severity of hepatic inflammation is determined prior to administration of the combination of (a) the compound of Formula (I), or a pharmaceutically acceptable salt thereof, and (b) an antioxidant, or a pharmaceutically acceptable salt thereof. In some embodiments, the severity of hepatic inflammation is determined prior to administration of the combination of (a) the compound of Formula (I), or a pharmaceutically acceptable salt thereof, and (b) an antioxidant, or a pharmaceutically acceptable salt thereof. In some embodiments, the severity of hepatic inflammation is determined during the period of time or after the period of time of administration of the combination of (a) and (b). In some embodiments, a decrease in the severity of hepatic inflammation during the period of time or after the period of time of administration of the combination of (a) and (b) compared to prior to administration of the combination of (a) and (b) indicates treatment of NAFLD. For example, a decrease in the severity of hepatic inflammation by about 1% to about 50%, about 25% to about 75%, or about 50% to about 100% indicates treatment of NAFLD. In some embodiments, a decrease in the severity of hepatic inflammation by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% indicates treatment of NAFLD.

In some embodiments, treatment of NAFLD comprises treatment of fibrosis and/or cirrhosis, e.g., a decrease in the severity of fibrosis, a lack of further progression of fibrosis and/or cirrhosis, or a slowing of the progression of fibrosis and/or cirrhosis. In some embodiments, the presence of fibrosis and/or cirrhosis is determined by one or more methods selected from the group consisting of transient elastography (e.g., FIBROSCAN®), non-invasive markers of hepatic fibrosis, and histological features of a liver biopsy. In some embodiments, the severity (e.g., stage) of fibrosis is determined by one or more methods selected from the group consisting of transient elastography (e.g., FIBROSCAN®), a fibrosis-scoring system, biomarkers of hepatic fibrosis (e.g., non-invasive biomarkers), and hepatic venous pressure gradient (HVPG). Non-limiting examples of fibrosis scoring systems include the NAFLD fibrosis scoring system (see, e.g., Angulo, et al., Hepatology. 2007; 45(4):846-54), the fibrosis scoring system in Brunt et al., Am J Gastroenterol. 1999, 94:2467-2474, the fibrosis scoring system in Kleiner et al., Hepatology. 2005, 41(6):1313-1321, and the ISHAK fibrosis scoring system (see Ishak et al., J Hepatol. 1995; 22:696-9), the contents of each of which are incorporated by reference herein in their entireties.

In some embodiments, the severity of fibrosis is determined prior to administration of the combination of (a) the compound of Formula (I), or a pharmaceutically acceptable salt thereof, and (b) an antioxidant, or a pharmaceutically acceptable salt thereof. In some embodiments, the severity of fibrosis is determined prior to administration of the combination of (a) the compound of Formula (I), or a pharmaceutically acceptable salt thereof, and (b) an antioxidant, or a pharmaceutically acceptable salt thereof. In some embodiments, the severity of fibrosis is determined during the period of time or after the period of time of administration of the combination of (a) and (b). In some embodiments, a decrease in the severity of fibrosis during the period of time or after the period of time of administration of the combination of (a) and (b) compared to prior to administration of the combination of (a) and (b) indicates treatment of NAFLD. In some embodiments, a decrease in the severity of fibrosis, a lack of further progression of fibrosis and/or cirrhosis, or a slowing of the progression of fibrosis and/or cirrhosis indicates treatment of NAFLD. In some embodiments, the severity of fibrosis is determined using a scoring system such as any of the fibrosis scoring systems described herein, for example, the score can indicate the stage of fibrosis, e.g., stage 0 (no fibrosis), stage 1, stage 2, stage 3, and stage 4 (cirrhosis) (see, e.g., Kleiner et al). In some embodiments, a decrease in the stage of the fibrosis is a decrease in the severity of the fibrosis. For example, a decrease by 1, 2, 3, or 4 stages is a decrease in the severity of the fibrosis. In some embodiments, a decrease in the stage, e.g., from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0 indicates treatment of NAFLD. In some embodiments, the stage of fibrosis decreases from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0 following administration of the combination of (a) and (b) compared to prior to administration of the combination of (a) and (b). In some embodiments, the stage of fibrosis decreases from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0 during the period of time of administration of the combination of (a) and (b) compared to prior to administration of the combination of (a) and (b). In some embodiments, the stage of fibrosis decreases from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0 after the period of time of administration of the combination of (a) and (b) compared to prior to administration of the combination of (a) and (b).

In some embodiments, the presence of NAFLD is determined by one or more biomarkers indicative of one or more of liver damage, inflammation, liver fibrosis, and/or liver cirrhosis or scoring systems thereof. In some embodiments, the severity of NAFLD is determined by one or more biomarkers indicative of one or more of liver damage, inflammation, liver fibrosis, and/or liver cirrhosis or scoring systems thereof. The level of the biomarker can be determined by, for example, measuring, quantifying, and monitoring the expression level of the gene or mRNA encoding the biomarker and/or the peptide or protein of the biomarker. Non-limiting examples of biomarkers indicative of one or more of liver damage, inflammation, liver fibrosis, and/or liver cirrhosis and/or scoring systems thereof include the aspartate aminotransferase (AST) to platelet ratio index (APRI); the aspartate aminotransferase (AST) and alanine aminotransferase (ALT) ratio (AAR); the FIB-4 score, which is based on the APRI, alanine aminotransferase (ALT) levels, and age of the subject (see, e.g., McPherson et al., Gut. 2010 September; 59(9):1265-9, which is incorporated by reference herein in its entirety); hyaluronic acid; pro-inflammatory cytokines; a panel of biomarkers consisting of α2-macroglobulin, haptoglobin, apolipoprotein A1, bilirubin, gamma glutamyl transpeptidase (GGT) combined with a subject's age and gender to generate a measure of fibrosis and necroinflammatory activity in the liver (e.g., FIBROTEST®, FIBROSURE®), a panel of biomarkers consisting of bilirubin, gamma-glutamyltransferase, hyaluronic acid, α2-macroglobulin combined with the subject's age and sex (e.g., HEPASCORE®; see, e.g., Adams et al., Clin Chem. 2005 October; 51(10):1867-73), and a panel of biomarkers consisting of tissue inhibitor of metalloproteinase-1, hyaluronic acid, and α2-macroglobulin (e.g., FIBROSPECT®); a panel of biomarkers consisting of tissue inhibitor of metalloproteinases 1 (TIMP-1), amino-terminal propeptide of type III procollagen (PIIINP) and hyaluronic acid (HA) (e.g., the Enhanced Liver Fibrosis (ELF) score, see, e.g., Lichtinghagen R, et al., J Hepatol. 2013 August; 59(2):236-42, which is incorporated by reference herein in its entirety). In some embodiments, the presence of fibrosis is determined by one or more of the FIB-4 score, a panel of biomarkers consisting of α2-macroglobulin, haptoglobin, apolipoprotein A1, bilirubin, gamma glutamyl transpeptidase (GGT) combined with a subject's age and gender to generate a measure of fibrosis and necroinflammatory activity in the liver (e.g., FIBROTEST®, FIBROSURE®), a panel of biomarkers consisting of bilirubin, gamma-glutamyltransferase, hyaluronic acid, α2-macroglobulin combined with the subject's age and sex (e.g., HEPASCORE®; see, e.g., Adams et al., Clin Chem. 2005 October; 51(10):1867-73), and a panel of biomarkers consisting of tissue inhibitor of metalloproteinase-1, hyaluronic acid, and α2-macroglobulin (e.g., FIBROSPECT®); and a panel of biomarkers consisting of tissue inhibitor of metalloproteinases 1 (TIMP-1), amino-terminal propeptide of type III procollagen (PIIINP) and hyaluronic acid (HA) (e.g., the Enhanced Liver Fibrosis (ELF) score).

In some embodiments, the level of aspartate aminotransferase (AST) does not increase. In some embodiments, the level of aspartate aminotransferase (AST) decreases. In some embodiments, the level of alanine aminotransferase (ALT) does not increase. In some embodiments, the level of alanine aminotransferase (ALT) decreases. In some embodiments, the “level” of an enzyme refers to the concentration of the enzyme, e.g., within blood. For example, the level of AST or ALT can be expressed as Units/L.

In some embodiments, the severity of fibrosis is determined by one or more of the FIB-4 score, a panel of biomarkers consisting of α2-macroglobulin, haptoglobin, apolipoprotein A1, bilirubin, gamma glutamyl transpeptidase (GGT) combined with a subject's age and gender to generate a measure of fibrosis and necroinflammatory activity in the liver (e.g., FIBROTEST®, FIBROSURE®), a panel of biomarkers consisting of bilirubin, gamma-glutamyltransferase, hyaluronic acid, α2-macroglobulin combined with the subject's age and sex (e.g., HEPASCORE®; see, e.g., Adams et al., Clin Chem. 2005 October; 51(10):1867-73, which is incorporated by reference herein in its entirety), and a panel of biomarkers consisting of tissue inhibitor of metalloproteinase-1, hyaluronic acid, and α2-macroglobulin (e.g., FIBROSPECT®); and a panel of biomarkers consisting of tissue inhibitor of metalloproteinases 1 (TIMP-1), amino-terminal propeptide of type III procollagen (PIIINP) and hyaluronic acid (HA) (e.g., the Enhanced Liver Fibrosis (ELF) score).

In some embodiments, hepatic inflammation is determined by the level of liver inflammation biomarkers, e.g., pro-inflammatory cytokines. Non-limiting examples of biomarkers indicative of liver inflammation include interleukin-(IL) 6, interleukin-(IL) 1β, tumor necrosis factor (TNF)-α, transforming growth factor (TGF)-β, monocyte chemotactic protein (MCP)-1, C-reactive protein (CRP), PAI-1, and collagen isoforms such as Col1a1, Col1a2, and Col4a1 (see, e.g., Neuman, et al., Can J Gastroenterol Hepatol. 2014 December; 28(11): 607-618 and U.S. Pat. No. 9,872,844, each of which are incorporated by reference herein in their entireties). Liver inflammation can also be assessed by change of macrophage infiltration, e.g., measuring a change of CD68 expression level. In some embodiments, liver inflammation can be determined by measuring or monitoring serum levels or circulating levels of one or more of interleukin-(IL) 6, interleukin-(IL) 10, tumor necrosis factor (TNF)-α, transforming growth factor (TGF)-β, monocyte chemotactic protein (MCP)-1, and C-reactive protein (CRP).

In some embodiments, the level of one or more biomarkers indicative of one or more of liver damage, inflammation, liver fibrosis, and/or liver cirrhosis is determined for a sample from the subject prior to administration of the combination of (a) the compound of Formula (I), or a pharmaceutically acceptable salt thereof, and (b) an antioxidant, or a pharmaceutically acceptable salt thereof. In some embodiments, the level of one or more biomarkers indicative of one or more of liver damage, inflammation, liver fibrosis, and/or liver cirrhosis is determined during the period of time or after the period of time of administration of the combination of (a) and (b). In some embodiments, a decrease in the level of one or more biomarkers indicative of one or more of liver damage, inflammation, liver fibrosis, and/or liver cirrhosis during the period of time or after the period of time of administration of the combination of (a) and (b) compared to prior to administration of the combination of (a) and (b) indicates treatment of NAFLD. For example, a decrease in the level of one or more biomarkers indicative of one or more of liver damage, inflammation, liver fibrosis, and/or liver cirrhosis by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% indicates treatment of NAFLD. In some embodiments, the decrease in the level of one or more biomarkers indicative of one or more of liver damage, inflammation, liver fibrosis, and/or liver cirrhosis following administration of the combination of (a) and (b) is by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%. In some embodiments, the level of one or more biomarkers indicative of one or more of liver damage, inflammation, liver fibrosis, and/or liver cirrhosis during the period of time of administration of the combination of (a) and (b) is by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%. In some embodiments, the level of one or more biomarkers indicative of one or more of liver damage, inflammation, liver fibrosis, and/or liver cirrhosis after the period of time of administration of the combination of (a) and (b) is by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%.

In some embodiments, the treatment of NAFLD decreases the level of serum bile acids in the subject. In some embodiments, the level of serum bile acids is determined by, for example, an ELISA enzymatic assay or the assays for the measurement of total bile acids as described in Danese et al., PLoS One. 2017; 12(6): e0179200, which is incorporated by reference herein in its entirety. In some embodiments, the level of serum bile acids can decrease by, for example, 10% to 40%, 20% to 50%, 30% to 60%, 40% to 70%, 50% to 80%, or by more than 90% of the level of serum bile acids prior to administration of (a) and (b). In some embodiments, the NAFLD is NAFLD with attendant cholestasis. In cholestasis, the release of bile, including bile acids, from the liver is blocked. Bile acids can cause hepatocyte damage (see, e.g., Perez M J, Briz O. World J Gastroenterol. 2009 Apr. 14; 15(14):1677-89) likely leading to or increasing the progression of fibrosis (e.g., cirrhosis) and increasing the risk of hepatocellular carcinoma (see, e.g., Sorrentino P et al., Dig Dis Sci. 2005 June; 50(6):1130-5 and Satapathy S K and Sanyal A J. Semin Liver Dis. 2015, 35(3):221-35, each of which are incorporated by reference herein in their entireties). In some embodiments, the NAFLD with attendant cholestasis is NASH with attendant cholestasis. In some embodiments, the treatment of NAFLD comprises treatment of pruritus. In some embodiments, the treatment of NAFLD with attendant cholestasis comprises treatment of pruritus. In some embodiments, a subject with NAFLD with attendant cholestasis has pruritus.

In some embodiments, treatment of NAFLD comprises an increase in adiponectin. It is thought that the compound of Formula (I) may be a selective activator of a highly limited number of PPARy pathways including pathways regulated by adiponectin. Adiponectin is an anti-fibrotic and anti-inflammatory adipokine in the liver (see e.g., Park et al., Curr Pathobiol Rep. 2015 Dec. 1; 3(4): 243-252). In some embodiments, the level of adiponectin is determined by, for example, an ELISA enzymatic assay. In some embodiments, the adiponectin level in the subject is increased by at least about 30%, at least about 68%, at least about 175%, or at least about 200%. In some embodiments, the increase is by at least about 175%. In some embodiments, the level of adiponectin is determined for a sample from the subject prior to administration of the combination of (a) the compound of Formula (I), or a pharmaceutically acceptable salt thereof, and (b) an antioxidant, or a pharmaceutically acceptable salt thereof. In some embodiments, the level of adiponectin is determined for a sample from the subject prior to administration of the combination of (a) the compound of Formula (I), or a pharmaceutically acceptable salt thereof, and (b) an antioxidant, or a pharmaceutically acceptable salt thereof. In some embodiments, the level of adiponectin is determined during the period of time or after the period of time of administration of the combination of (a) and (b). In some embodiments, an increase in the level of adiponectin during the period of time or after the period of time of administration of the combination of (a) and (b) compared to prior to administration of the combination of (a) and (b) indicates treatment of NAFLD. For example, an increase in the level of adiponectin by at least about 30%, at least about 68%, at least about 175%, or at least about 200% indicates treatment of NAFLD. In some embodiments, the increase in the level of adiponectin following administration of the combination of (a) and (b) is at least about 200%.

Provided herein are methods of treating non-alcoholic fatty liver disease (NAFLD) in a subject in need thereof comprising or consisting essentially of administering to the subject (a) the compound of Formula (I), or a pharmaceutically acceptable salt thereof, and (b) an antioxidant, or a pharmaceutically acceptable salt thereof, wherein the amounts of (a) and (b) together are effective in treating NAFLD. In some embodiments, a method of treating non-alcoholic fatty liver disease (NAFLD) in a subject in need thereof comprises or consists essentially of administering to the subject (a) the compound of Formula (I), or a pharmaceutically acceptable salt thereof, and (b) an antioxidant, or a pharmaceutically acceptable salt thereof, during a period of time, wherein the amounts of (a) and (b) together are effective in treating NAFLD.

Also provided herein are methods of treating fibrosis in a subject in need thereof comprising or consisting essentially of administering to the subject (a) the compound of Formula (I), or a pharmaceutically acceptable salt thereof, and (b) an antioxidant, or a pharmaceutically acceptable salt thereof, wherein the amounts of (a) and (b) together are effective in treating fibrosis. In some embodiments, a method of treating fibrosis in a subject in need thereof comprises or consists essentially of administering to the subject (a) the compound of Formula (I), or a pharmaceutically acceptable salt thereof, and (b) an antioxidant, or a pharmaceutically acceptable salt thereof, during a period of time, wherein the amounts of (a) and (b) together are effective in treating fibrosis.

Also provided herein are methods of treating steatosis in a subject in need thereof comprising or consisting essentially of administering to the subject (a) the compound of Formula (I), or a pharmaceutically acceptable salt thereof, and (b) an antioxidant, or a pharmaceutically acceptable salt thereof, wherein the amounts of (a) and (b) together are effective in treating steatosis. In some embodiments, a method of treating steatosis in a subject in need thereof comprises or consists essentially of administering to the subject (a) the compound of Formula (I), or a pharmaceutically acceptable salt thereof, and (b) an antioxidant, or a pharmaceutically acceptable salt thereof, during a period of time, wherein the amounts of (a) and (b) together are effective in treating steatosis.

Also provided herein are methods of treating a subject, the method comprising: selecting a subject having non-alcoholic fatty liver disease (NAFLD); and administering (a) the compound of Formula (I), or a pharmaceutically acceptable salt thereof, and (b) an antioxidant, or a pharmaceutically acceptable salt thereof, to the selected subject, wherein the amounts of (a) and (b) together are effective in treating NAFLD. In some embodiments, (a) and (b) are administered during a period of time.

Also provided herein are methods of treating a subject, the method comprising: identifying a subject having non-alcoholic fatty liver disease (NAFLD); and administering (a) the compound of Formula (I), or a pharmaceutically acceptable salt thereof, and (b) an antioxidant, or a pharmaceutically acceptable salt thereof, to the selected subject, wherein the amounts of (a) and (b) together are effective in treating NAFLD. In some embodiments, (a) and (b) are administered during a period of time.

Also provided herein are methods of selecting a subject for participation in a clinical trial, the method comprising: identifying a subject having NAFLD; and selecting the identified subject for participation in a clinical trial that comprises administration of (a) a therapeutically effective amount of the compound of Formula (I), or a pharmaceutically acceptable salt thereof, (b) a therapeutically effective amount of an antioxidant, or a pharmaceutically acceptable salt thereof, or a pharmaceutically acceptable salt thereof. In some embodiments, the amounts of (a) and (b) together are effective in treating NAFLD.

In some embodiments, (a) and (b) are administered concurrently. In some embodiments, (a) and (b) are administered as a fixed combination. In some embodiments, (a) and (b) are administered as a non-fixed combination. In some embodiments, (a) and (b) are administered sequentially and in any order, at specific or varying time intervals (e.g., during the period of time). In some embodiments, a therapeutically effective amount of each of (a) and (b) are administered concurrently. In some embodiments, a therapeutically effective amount of each of (a) and (b) are administered sequentially and in any order, at specific or varying time intervals (e.g., during the period of time).

In some embodiments, the amount of the compound of Formula (I), or a pharmaceutically acceptable salt thereof, is from about 0.1 to about 15 milligrams (mg), or any value in between. For example, from about 0.1 to about 10 mg, about 5 to about 15 mg, or about 2 to about 12 mg. In some embodiments, the compound of Formula (I), or a pharmaceutically acceptable salt thereof, is administered at a dose of about 0.5 mg, 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 12.5 mg, 13.0 mg, 13.5 mg, 14.0 mg, 14.5 mg, or 15.0 mg. In some embodiments, the dose is a therapeutically effective amount.

In some embodiments, the compound of Formula (I), or a pharmaceutically acceptable salt thereof, is administered to the subject twice a day, daily, every other day, three times a week, twice a week, weekly, every other week, twice a month, or monthly. In some embodiments, the compound of Formula (I), or a pharmaceutically acceptable salt thereof, is administered to the subject daily.

In some embodiments, the compound of Formula (I), or a pharmaceutically acceptable salt thereof, is administered to the subject daily and at a dose of about 3 mg. In some embodiments, the compound of Formula (I), or a pharmaceutically acceptable salt thereof, is administered at a dose from about 0.1 to about 10.0 mg per day. In some embodiments, the compound of Formula (I), or a pharmaceutically acceptable salt thereof, is administered at a dose from about 0.1 to about 3 mg per day. In some embodiments, the compound of Formula (I), or a pharmaceutically acceptable salt thereof, is administered at a dose about 0.5 mg per day. In some embodiments, the compound of Formula (I), or a pharmaceutically acceptable salt thereof, is administered at a dose about 1 mg per day. In some embodiments, the compound of Formula (I), or a pharmaceutically acceptable salt thereof, is administered at a dose about 2 mg per day.

In some of any of the above embodiments, the compound of Formula (I) is in the form of a besylate salt. In some embodiments, the compound of Formula (I) is in the form of an HCl salt. In some embodiments, the compound of Formula (I) is in the form of an HBr salt. In some embodiments, the compound of Formula (I) is in the form of a tosylate salt.

In some embodiments, the antioxidant, or a pharmaceutically acceptable salt thereof, is selected from the group consisting of: a vitamin, a mineral, a polyphenol, an anthocyanin, a carotenoid, or a glucosinolate, or a pharmaceutically acceptable salt thereof. In some embodiments, the antioxidant is selected from the group consisting of:

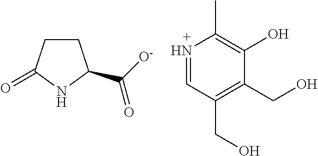

(metadoxine), vitamin A, vitamin B1, vitamin B2, vitamin B3, vitamin B5, vitamin B6, vitamin B12, folic acid, vitamin C, vitamin D, vitamin E, selenium, N-acteylcysteine, pyrrolidinedithiocarbamate,

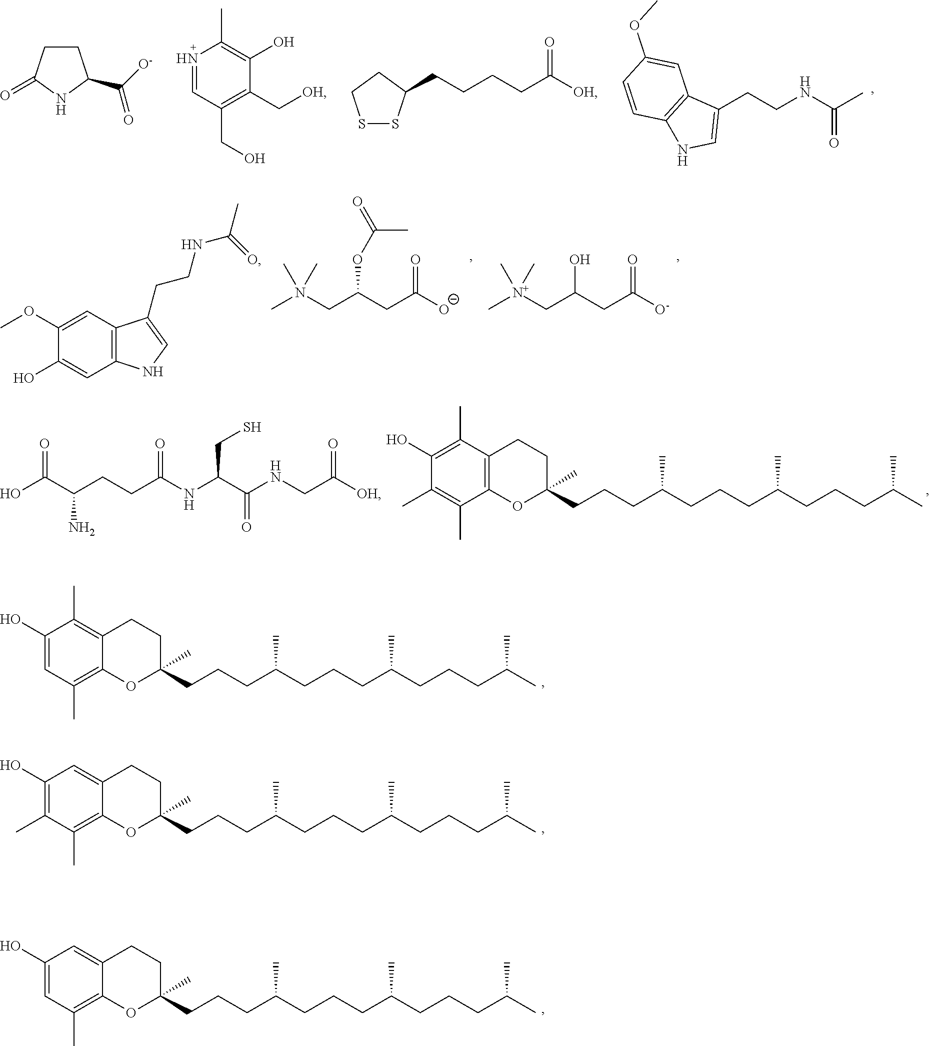

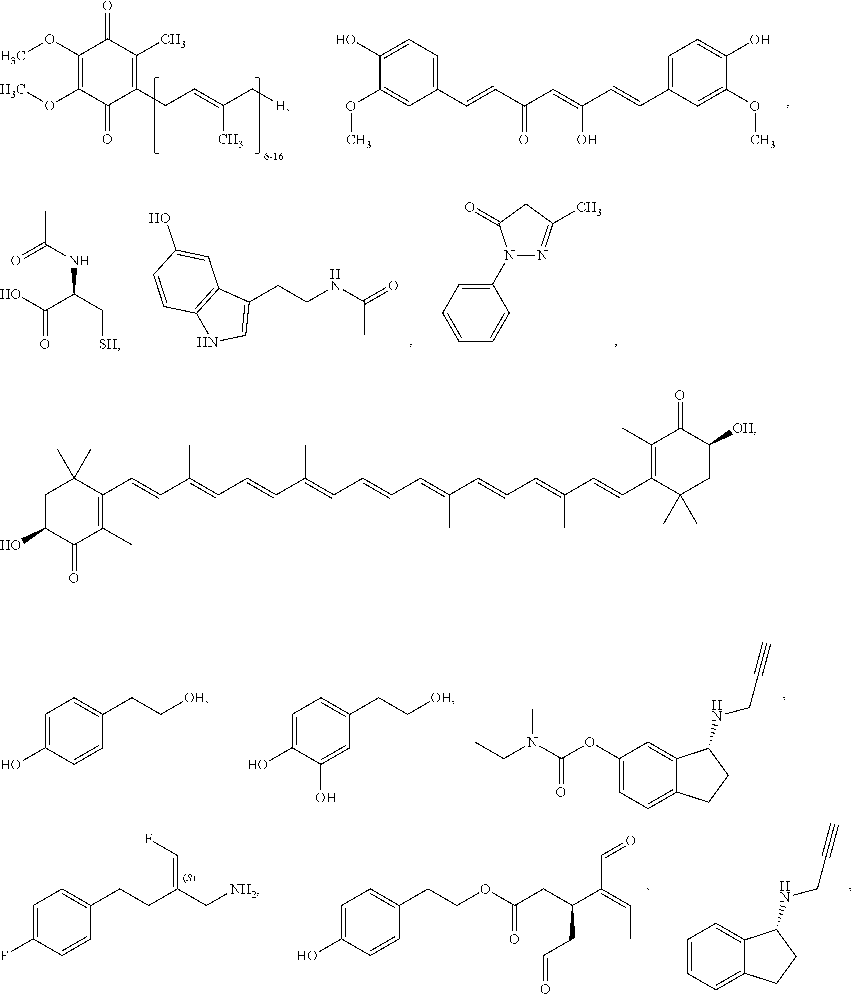

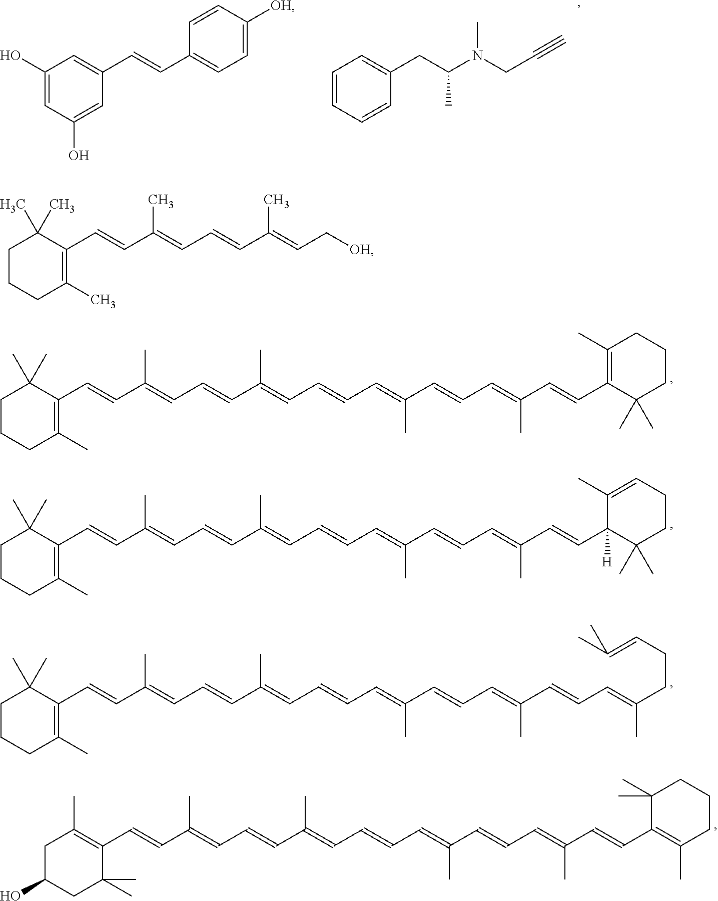

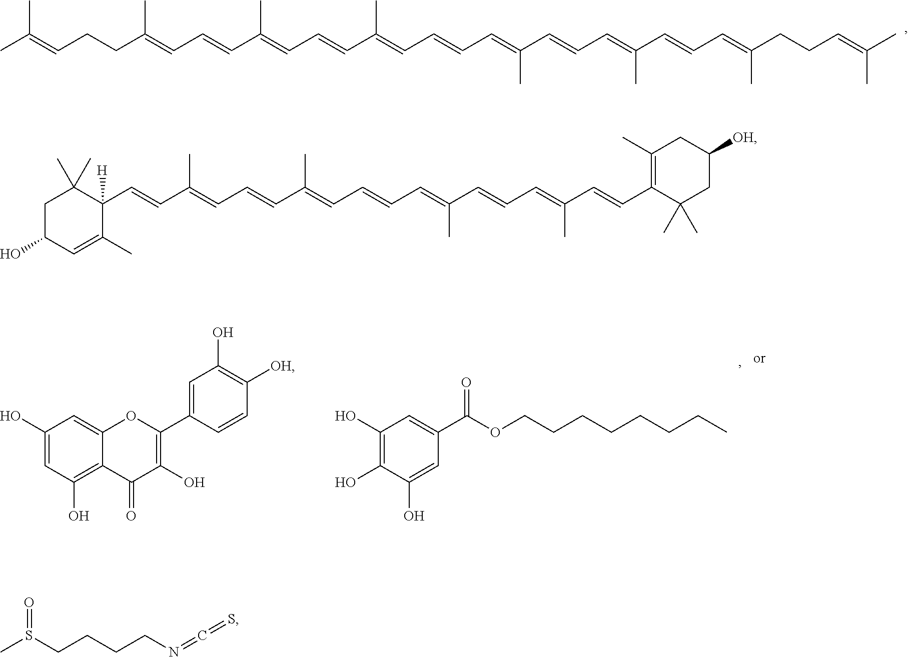

or a pharmaceutically acceptable salt or combination of any of the foregoing. In some embodiments, the antioxidant is a vitamin.

In some embodiments, the antioxidant, or a pharmaceutically acceptable salt thereof, is a vitamin selected from the group consisting of: vitamin A, vitamin B1, vitamin B2, vitamin B3, vitamin B5, vitamin B6, vitamin B12, vitamin C, vitamin D, and vitamin E. In some embodiments, the antioxidant is not a vitamin. In some embodiments, the antioxidant is not any of vitamin A, vitamin B1, vitamin B2, vitamin B3, vitamin B5, vitamin B6, vitamin B12, vitamin C, vitamin D, or vitamin E. In some embodiments, the antioxidant is not vitamin E.

In some embodiments, the amount of the compound of Formula (I), or a pharmaceutically acceptable salt thereof, is from about 1 to about 350 mg, or any value in between. For example, about 1 to about 175 mg, about 175 to about 350 mg, about 90 to about 260 mg, or about 150 to 200 mg.

In some embodiments, the amount of the antioxidant, or a pharmaceutically acceptable salt thereof, is from about 1 mg to about 2 g, or any value in between. For example, about 1 mg to about 4 mg, about 2 mg to about 25 mg, about 10 mg to about 50 mg, about 30 mg to about 75 mg, about 50 mg to about 100 mg, about 100 to about 250 mg, about 250 to about 500 mg, about 500 to about 1 g, about 1 to about 1.5 g, or about 1.5 to 2 g. In some embodiments, the amount of the antioxidant is between about 1 mg and about 4 mg. In other embodiments, the amount of the antioxidant is between about 50 mg and about 60 mg.

In some embodiments, the antioxidant is administered at the USFDA's recommended daily allowance (RDA). In some embodiments, the antioxidant is administered in an amount about 25% to about 500% of the RDA, for example, about 25%, about 30%, about 35%, about 40%, about 45%, about 50%, about 75%, about 100%, about 125%, about 150%, about 175%, about 200%, about 225%, about 250%, about 275%, about 300%, about 325%, about 350%, about 375%, about 400%, about 425%, about 450%, about 475%, or about 500%. In some embodiments, the antioxidant is administered in an amount about 25% to about 35% of the RDA. In other embodiments, the antioxidant is administered in an amount about 250% to about 350% of the RDA.

In some embodiments, the amount of the antioxidant, or a pharmaceutically acceptable salt thereof, is less than 5 mg (e.g., 1 mg, 2 mg, 3 mg, or 4 mg). In some embodiments, the amount of the antioxidant, or a pharmaceutically acceptable salt thereof, is more than 30 mg, for example, from above 30 mg to about 500 mg.

In some embodiments, the antioxidant, or a pharmaceutically acceptable salt thereof, is administered to the subject twice a day, daily, every other day, three times a week, twice a week, weekly, every other week, twice a month, or monthly. In some embodiments, the antioxidant, or a pharmaceutically acceptable salt thereof, is administered to the subject daily.

In some embodiments, treatment of NAFLD comprises a decrease of one or more symptoms associated with NAFLD in the subject. Exemplary symptoms can include one or more of an enlarged liver, fatigue, pain in the upper right abdomen, abdominal swelling, enlarged blood vessels just beneath the skin's surface, enlarged breasts in men, enlarged spleen, red palms, jaundice, and pruritus. In some embodiments, the subject is asymptomatic.

In some embodiments, the treatment of NAFLD, e.g., NAFL or NASH, comprises a reduction in hepatic steatosis. For example, hepatic steatosis is decreased by at least 2%, 3%, 4%, 5%, 6%, 7%, 8%. 9%, 10, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 96%, 97%, 98%, 99%, or more than 99% following administration of (a) and (b) for a period of time.

In some embodiments, the treatment of NAFLD, e.g., NAFL or NASH, is assessed using the NAFLD Activity Score (NAS). In some embodiments, treatment of NAFLD comprises a decrease in the NAS. In some embodiments, the NAS for a sample from the subject following administration is 7 or less. In some embodiments, the NAS for a sample from the subject following administration is 5 or less, 4 or less, 3 or less, or 2 or less. In some embodiments, the NAFLD activity score (NAS) for a sample from the subject following administration during the period of time is 7 or less. In some embodiments, the NAS for a sample from the subject following administration during the period of time is 5 or less, 4 or less, 3 or less, or 2 or less. In some embodiments, the sample from the subject is from a liver biopsy.

In some embodiments, the treatment of NAFLD, e.g., NAFL or NASH, can be assessed using the NAFLD Activity Score (NAS). In some embodiments, the NAS for a sample from the subject following administration is reduced by 1 or more, 2 or more, 3 or more, 4 or more, 5 or more, or 6 or more. In some embodiments, the NAS for a sample from the subject following administration is reduced by 1, 2, 3, 4, 5, or 6. In some embodiments, the NAFLD activity score (NAS) for a sample from the subject following administration during the period of time is reduced by 1 or more, 2 or more, 3 or more, 4 or more, 5 or more, or 6 or more. In some embodiments, the NAS for a sample from the subject following administration during the period of time is reduced by 1, 2, 3, 4, 5, or 6. In some embodiments, the sample from the subject is from a liver biopsy.

In some embodiments, the treatment of NAFLD, e.g., NAFL or NASH, comprises treatment of hepatic inflammation. In some embodiments, the severity of the hepatic inflammation is decreased by about 1% to about 50%, about 25% to about 75%, or about 50% to about 100%. In some embodiments, the severity of hepatic inflammation is decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

In some embodiments, the treatment of NAFLD, e.g., NAFL or NASH, comprises treatment of fibrosis. In some embodiments, the treatment of the NAFLD comprises treatment of cirrhosis (e.g., stage 4 of fibrosis). In some embodiments, treatment of fibrosis comprises a decrease in the stage of fibrosis, for example, from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0.

In some embodiments, the adiponectin level in the subject is increased by at least about 30%, at least about 68%, at least about 175%, or at least about 200%. In some embodiments, the increase is by at least about 175%.

In some embodiments, the level of aspartate aminotransferase (AST) in the subject does not increase. In some embodiments, the level of aspartate aminotransferase (AST) in the subject decreases. In some embodiments, the level of alanine aminotransferase (ALT) in the subject does not increase. In some embodiments, the level of alanine aminotransferase (ALT) in the subject decreases. In some embodiments, the total body weight of the subject does not increase. In some embodiments, the total body weight of the subject decreases. In some embodiments, the body mass index (BMI) of the subject does not increase. In some embodiments, the body mass index (BMI) of the subject decreases. In some embodiments, the waist and hip (WTH) ratio of the subject does not increase. In some embodiments, the waist and hip (WTH) ratio of the subject decreases.

In some embodiments, a non-invasive liver fibrosis marker does not increase or decreases. In some embodiments, the non-invasive liver fibrosis marker is Enhanced Liver Fibrosis (ELF) panel.

In some embodiments, treatment of NAFLD comprises a decrease in the level of one or more biomarkers indicative of one or more of liver damage, inflammation, fibrosis, and/or cirrhosis, e.g., any of the biomarkers as described herein. In some embodiments, treatment of NAFLD comprises a decrease in the level of one or more biomarkers indicative of one or more of liver damage, inflammation, fibrosis, and/or cirrhosis by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%.

In some embodiments, the treatment of NAFLD decreases the level of serum bile acids in the subject. In some embodiments, the treatment of NAFLD comprises treatment of pruritus.

In some embodiments, the subject has liver fibrosis associated with the NAFLD. In some embodiments, the subject has hepatic cirrhosis (e.g., stage 4 fibrosis) associated with the NAFLD. In some embodiments, the subject has liver fibrosis as a comorbidity. In some embodiments, the subject has hepatic cirrhosis (e.g., stage 4 fibrosis) as a comorbidity. In some embodiments, the subject has liver fibrosis caused by the NAFLD. In some embodiments, the subject has hepatic cirrhosis (e.g., stage 4 fibrosis) caused by the NAFLD.

In some embodiments, the NAFLD is simple nonalcoholic fatty liver (NAFL). In some embodiments, the NAFLD is NAFL with attendant liver fibrosis. In some embodiments, the NAFLD is NAFL with attendant liver cirrhosis.

In some embodiments, the NAFLD is nonalcoholic steatohepatitis (NASH). In some embodiments, the NAFLD is NASH with attendant liver fibrosis. In some embodiments, the NAFLD is NASH with attendant liver cirrhosis.

In some embodiments, the method further comprises performing a liver biopsy to determine the NAFLD activity score of the biopsy sample obtained from the subject.

In some embodiments, (a) and (b) are administered prophylactically.

In some embodiments, the subject was previously treated, before the period of time, with one or more therapeutic agents, e.g., treatment with at least one NAFLD treatment, NASH treatment, type 2 diabetes treatment, obesity treatment, metabolic syndrome treatment, liver disease treatment, cardiovascular treatment, heart failure treatment, hypertension treatment. In some embodiments, the one or more therapeutic agents that were administered to the patient before the period of time was unsuccessful (e.g., therapeutically unsuccessful as determined by a physician). In some embodiments, the unsuccessful treatment did not comprises or consist essentially of administration of (a) and (b).

In some embodiments, the subject has Type I diabetes as a comorbidity. In other embodiments, the subject has Type II diabetes as a comorbidity. In some embodiments, the subject has adequate glycemic control, prior to receiving the combination of (a) and (b). For example, in some embodiments, the subject has an HbA_{1c} level of ≤10%, or ≤9%, or ≤8%, or ≤7%, or ≤6%, or ≤5%, or ≤4%, or any value in between, prior to receiving the combination of (a) and (b). In some embodiments, the subject has an HbA_{1c} level of about 4% to about 6%, prior to receiving the combination of (a) and (b). In other embodiments, the subject has an HbA_{1c} level of about 5% to about 8%, prior to receiving the combination of (a) and (b). In still other embodiments, the subject has an HbA_{1c} level of about 6% to about 10%, prior to receiving the combination of (a) and (b).

In some embodiments, the subject's HbA_{1c} level decreases by about 1% to about 5% after receiving the combination of (a) and (b); for example, about 1% to about 2%, about 1.5% to about 2.5%, about 2% to about 3%, about 2.5% to about 3.5%, about 3% to about 4%, about 3.5% to about 4.5%, about 4% to about 5%, or about 1.5% to about 3%, or any value in between. In some embodiments, the subject's HbA_{1c} level decreases by about 1.5% to about 3% after receiving the combination of (a) and (b). In some embodiments, the subject does not have Type I diabetes as a comorbidity. In other embodiments, the subject does not have Type II diabetes as a comorbidity.

In some embodiments, the subject has a mean fasting plasma glucose level of ≤170 mg/dL, ≤160 mg/dL, ≤150 mg/dL, ≤140 mg/dL, ≤130 mg/dL, ≤120 mg/dL, 110 mg/dL, or ≤100 mg/dL. In some embodiments, the subject has a mean fasting plasma glucose level, prior to receiving the combination of (a) and (b), of about 90 mg/dL to about 110 mg/dL. In other embodiments, the subject has a mean fasting plasma glucose level, prior to receiving the combination of (a) and (b), of about 100 mg/dL to about 120 mg/dL. In still other embodiments, the subject has a mean fasting plasma glucose level, prior to receiving the combination of (a) and (b), of about 110 mg/dL to about 130 mg/dL. In some other embodiments, the subject has a mean fasting plasma glucose level, prior to receiving the combination of (a) and (b), of about 120 mg/dL to about 140 mg/dL. In some embodiments, the subject has a mean fasting plasma glucose level, prior to receiving the combination of (a) and (b), of about 130 mg/dL to about 150 mg/dL. In other embodiments, the subject has a mean fasting plasma glucose level, prior to receiving the combination of (a) and (b), of about 140 mg/dL to about 160 mg/dL. In still other embodiments, the subject has a mean fasting plasma glucose level, prior to receiving the combination of (a) and (b), of about 150 mg/dL to about 170 mg/dL. In some embodiments, the subject's mean fasting plasma glucose level decreases by about 30 mg/dL to about 90 mg/dL after receiving the combination of (a) and (b); for example, by about 30 mg/dL to about 40 mg/dL, about 40 mg/dL to about 50 mg/dL, about 50 mg/dL to about 60 mg/dL, about 60 mg/dL to about 70 mg/dL, about 70 mg/dL to about 80 mg/dL, or about 80 mg/dL to about 90 mg/dL, or any value in between.

In some embodiments, the subject has a BMI of ≤35, ≤34, ≤33, ≤32, ≤31, ≤30, ≤29, ≤28, ≤27, ≤26, ≤25, ≤24, ≤23, ≤22, ≤21, or ≤20, or any value in between, prior to receiving the combination of (a) and (b). In some embodiments, the subject has a BMI of about 35 to about 40, prior to receiving the combination of (a) and (b). In other embodiments, the subject has a BMI of about 32 to about 35, prior to receiving the combination of (a) and (b). In still other embodiments, the subject has a BMI of about 28 to about 32, prior to receiving the combination of (a) and (b). In some other embodiments, the subject has a BMI of about 26 to about 30, prior to receiving the combination of (a) and (b). In yet other embodiments, the subject has a BMI of about 24 to about 28, prior to receiving the combination of (a) and (b). In some embodiments, the subject has a BMI of about 22 to about 26, prior to receiving the combination of (a) and (b). In other embodiments, the subject has a BMI of about 20 to about 24, prior to receiving the combination of (a) and (b). In some embodiments, the subject's BMI changes from about −10% to about +10% after receiving the combination of (a) and (b). In some embodiments, the subject's BMI decreases by about 0% to about 10% after receiving the combination of (a) and (b). In some embodiments, the subject's BMI decreases by about 0.5% to about 5% after receiving the combination of (a) and (b). In some embodiments, the decrease in the subject's BMI occurs within about 4 weeks to about 104 weeks; for example, about 4 weeks to about 8 weeks, about 6 weeks to about 12 weeks, about 8 weeks to about 16 weeks, about 12 weeks to about 24 weeks, about 16 weeks to about 40 weeks, about 24 weeks to about 52 weeks, about 32 weeks to about 64 weeks, about 40 weeks to about 80 weeks, about 52 weeks to about 96 weeks, about 72 weeks to about 104 weeks, or any value in between.

In some embodiments, the subject's weight changes from about −10% to about +10% after receiving the combination of (a) and (b). In some embodiments, the subject's weight changes from about −5% to about +5% after receiving the combination of (a) and (b). In some embodiments, the subject's weight decreases by about 0% to about 10% after receiving the combination of (a) and (b). In some embodiments, the subject's weight decreases by about 0.5% to about 5% after receiving the combination of (a) and (b). In some embodiments, the subject's weight changes from about −5 kg to about +5 kg after receiving the combination of (a) and (b). In some embodiments, the subject's weight changes from about −2 kg to about +2 kg after receiving the combination of (a) and (b). In some embodiments, the subject's weight decreases by about 0 kg to about 5 kg after receiving the combination of (a) and (b). In some embodiments, the subject's weight decreases by about 0.5 kg to about 2 kg after receiving the combination of (a) and (b). In some embodiments, the changes in the subject's weight occurs within about 4 weeks to about 104 weeks; for example, about 4 weeks to about 8 weeks, about 6 weeks to about 12 weeks, about 8 weeks to about 16 weeks, about 12 weeks to about 24 weeks, about 16 weeks to about 40 weeks, about 24 weeks to about 52 weeks, about 32 weeks to about 64 weeks, about 40 weeks to about 80 weeks, about 52 weeks to about 96 weeks, about 72 weeks to about 104 weeks, or any value in between.

### EXAMPLES

The following examples further illustrate the invention. For example, the efficacy of CHS-131, alone or in combination with an antioxidant, or a pharmaceutically acceptable salt thereof, to treat NAFLD is demonstrated in the following examples.

### Example 1

The effects of treatment with CHS-131 (Compound of Formula (I)), alone and in combination with other therapeutic agents, to treat NASH are evaluated in mice. Various models can be used. Subjects are divided into groups for treatment and evaluation. Groups can include, controls (e.g. subjects on or off diets that are not administered a therapy), subjects administered monotherapy (e.g. CHS-131, antioxidant), subjects administered a combo-therapy (e.g. CHS-131 and antioxidant), and subjects administered a positive control therapy. Metabolic parameters, hepatic pathology, and NAFLD Activity Score including fibrosis stage are evaluated. Each animal is administered the respective compositions (e.g. vehicle, monotherapy, combo-therapy) starting on Day 0 and ending on Day 82-84. Samples, as described in Table 2, are collected for analysis.

TABLE 2
Samples collected over course of study

| Sample | Usage | Groups | Time Point | Method |
|---|---|---|---|---|
| Liver pre-biopsy | Stratification and | All | 3 weeks | Dissection |
| | randomization, NAFLD | | before start | |
| | Activity Score, Fibrosis | | of study | |
| | Stage, Col1a1 | | | |
| BG Baseline | Blood Glucose | All | 1 week | Tail Vein |
| | | | before start | |
| | | | of study | |
| Plasma insulin baseline | Plasma insulin | All | 1 week | Tail Vein |
| | | | before start | |
| | | | of study | |
| OGTT | Blood Glucose | All | Week 7-8 | Tail Vein |
| IPTT | Blood Glucose | All | Week 9/10 | Tail Vein |
| BG week 12 | Blood Glucose | All | Week 12 | Tail Vein |
| Plasma insulin week 12 | Plasma insulin | All | Week 12 | Tail Vein |
| Terminal | ALT/AST/TG/TC/BUN | All | Week 12 | Tail Vein |
| ALT/AST/TG/ | | | | |
| TC/BUN/creatinine | | | | |
| Liver post-biopsy | NAFLD Activity Score, | All | Termination | Dissection |
| | and fibrosis stage and | | | |
| | steatosis stage and Col1a1 | | | |
| | and Galectin-3 and a-SMA | | | |
| | quantification | | | |
| Liver TG/TC | Liver triglyceride and total | All | Termination | Dissection |
| | cholesterol | | | |
| Liver HP | Liver hydroxyproline | All | Termination | Dissection |
| Liver RNA | RNAseq (optional) | All | Termination | Dissection |
| Liver | Evaluation | All | Termination | Dissection |
| Muscle | Evaluation | All | Termination | Dissection |
| Epididymal fat | Evaluation | All | Termination | Dissection |
| Subcutaneous fat | Evaluation | All | Termination | Dissection |
| Kidney | Evaluation | All | Termination | Dissection |
| Brain | Evaluation | All | Termination | Dissection |
| Heart | Evaluation | All | Termination | Dissection |
| Terminal plasma | Evaluation | All | Termination | Cardiac |
| | | | | Puncture |

ALT is alanine transaminase; a-SMA is alpha-smooth muscle actin; AST is aspartate transaminase; BG is blood glucose; BUN is blood urea nitrogen; Col1a1 is collagen 1a1; OGTT is oral glucose tolerance test; WPITT is intraperitoneal insulin tolerance test; TG is triglycerides; TC is total cholesterol; HP is hydroxyproline

An overview of sample analyses that are performed during the study are listed in Tables 3-5, below.

TABLE 3
In vivo pharmacology

| Analysis Name | Groups | Samples | Period or Frequency | Comments |
|---|---|---|---|---|
| Bodyweight | All | Whole | QD | na |
| | | animal | | |
| Food intake | All | Whole | QD week 0 + 1 | AM |
| | | animal | QW (24 h) | |
| | | | Week 2-12 | |
| Echo MRI | All | Whole | Week 1 | na |
| baseline | | animal | | |
| Echo MRI | All | Whole | Week 11 | na |
| week 11 | | animal | | |
| Liver weight | All | Whole liver | Termination | na |
| | | weight (wet | | |
| | | weight) | | |

TABLE 4
Histology

| Analysis Name | Groups | Samples | Comments |
|---|---|---|---|
| Fibrosis stage | All | Liver pre-biopsy | PSR staining |
| | | Liver post-biopsy | Re-staining of |
| | | | pre-biopsy |
| NAFLD Activity | All | Liver pre-biopsy | HE staining |
| Score | | Liver post-biopsy | Re-staining of |
| | | | pre-biopsy |
| Col1a1 | All | Liver pre-biopsy | IHC (randomization) |
| | | Liver post-biopsy | IHC |
| Galectin-3 | All | Liver post-biopsy | IHC |
| quantification | | | |
| Steatosis quantification | All | Liver post-biopsy | HE staining |
| a-SMA quantification | All | Liver post-biopsy | IHC |

TABLE 5
Assays

| Analysis Name | Groups | Samples |
|---|---|---|
| Plasma insulin | All | Plasma insulin baseline |
| baseline | | |
| Plasma insulin | All | Plasma insulin baseline |
| week 12 | | |
| Plasma ALT | All | Terminal ALT/AST/TG/TC/BUN/Creatinine |
| Plasma AST | All | Terminal ALT/AST/TG/TC/BUN/Creatinine |
| Plasma TG | All | Terminal ALT/AST/TG/TC/BUN/Creatinine |
| Plasma TC | All | Terminal ALT/AST/TG/TC/BUN/Creatinine |
| Plasma BUN | All | Terminal ALT/AST/TG/TC/BUN/Creatinine |
| Plasma creatinine | All | Terminal ALT/AST/TG/TC/BUN/Creatinine |
| Liver triglycerides | All | Liver TG/TC |
| Liver total | All | Liver TG/TC |
| cholesterol | | |
| Liver | All | Liver HP |
| hydroxyproline | | |

NAFLD Activity Score (NAS) and Fibrosis stage are evaluated as follows. Liver samples are fixed in formalin, paraffin embedded and sections are stained with hematoxylin and eosin (H&E) and Sirius Red. Samples are scored for NAS and fibrosis stage (outlined below) using of the clinical criteria outlined by Kleiner et al. 2005. Total NAS score represents the sum of scores for steatosis, inflammation, and ballooning, and ranges from 0-8.

Total NAS scoring

| Feature | Degree | Score |
|---|---|---|
| Steatosis | <5% | 0 |
| | 5-33% | 1 |
| | >33-66% | 2 |
| | >66% | 3 |
| Lobular | No foci | 0 |
| inflammation | <2 foci/200x | 1 |
| | 2-4 foci/200x | 2 |
| | >4 foci/200x | 3 |
| Ballooning | None | 0 |
| degeneration | Few | 1 |
| | Many cells/prominent ballooning | 2 |
| Fibrosis | None | 0 |
| | Perisinusoidal or periportal | 1 |
| | Perisinusoidal & portal/periportal | 2 |
| | Bridging fibrosis | 3 |
| | Cirrhosis | 4 |

Adopted from: Design and validation of a histological scoring system for non-alcoholic fatty liver disease, Kleiner et al., Hepatology 41; 2005.

For lobular inflammation, inflammation is evaluated by counting the number of inflammatory foci per field using a 200× magnification (min. 5 fields per animal). A focus is defined as a cluster, not a row, of >3 inflammatory cells. Acidophil bodies are not included in this assessment, nor is portal inflammation. Fibrosis stage is evaluated separately from NAS.

### IHC and Steatosis Quantification

Quantitative assessment of immunoreactivity is evaluated as follows. IHC-positive staining is quantified by image analysis using the Visiomorph software (Visiopharm, Denmark). Visiomorph protocols are designed to analyze the virtual slides in two steps: 1. Crude detection of tissue at low magnification (1× objective). The liver capsule is excluded. 2. Detection of IHC-positive staining (e.g., green; collagen 1 IHC), tissue (e.g., red) and fat (e.g., pink) at high magnification (10× objective). The quantitative estimate of IHC-positive staining is calculated as an area fraction (AF) according to the following formula:

$$A {F}_{IHC - po s} = \frac{A r e {a}_{IHC - pos .}}{A r e {a}_{f a t} + A r e {a}_{t i s s u e} + A r e {a}_{IHC - p o s}}$$

Quantitative assessment of steatosis is evaluated as follows. Steatosis is quantified on H&E stained slides by image analysis using the Visiomorph software (Visiopharm, Denmark). Visiomorph protocols are designed to analyse the virtual slides in two steps: 1. Crude detection of tissue at low magnification (1× objective). 2. Detection of steatosis (pink) and tissue (blue) at high magnification (20× objective). The quantitative estimate of steatosis is calculated as an area fraction (AF) according to the following formula:

$$A {F}_{s t e a t o s i s} = \frac{A r e {a}_{s t e a t o s i s}}{A r e {a}_{t i s s u e} + A r e {a}_{s t e a t o s i s}}$$

### Example 2—Effect of 12 Weeks of CHS-131 Alone and in Combination with an Antioxidant on Liver Disease in Pre-Biopsied Male Ob/Ob-NASH Mice

This study assesses the effects of treatment with CHS-131 (Compound of Formula (I)), alone and in combination with other therapeutic agents, to treat NASH. Metabolic parameters, hepatic pathology, and NAFLD Activity Score including fibrosis stage are evaluated in ob/ob mice. In addition to the description below, this study may include sample collection, testing, measurement, and evaluation (e.g. histology, biochemical, gene expression, genetic), and analysis as described in the examples above.

ob/ob mice are homozygous for a spontaneous Lep^{ob} point mutation in the gene encoding leptin and are consistently fibrosis prone when cholesterol (2%) and trans-fatty acids (45% of total fat amount) are added to a high-caloric diet. These mice will develop steatohepatitis and fibrosis within a shorter timeframe (≤12 weeks) compared with wild-type C57BL/6 mice fed the same diet (≥26 weeks). See, e.g., Kristiansen, et al., World J. Hepatol., Vol. 8, pp. 673-684 (2016). The ob/ob mice also display a more significant insulin resistant and NASH phenotype than the high-caloric diet, well suited for evaluating potential anti-NASH therapeutics. Protocols for evaluating treatment of NASH in mouse models are found in Tolbol, et al., World J Gastroenterol. 2018 Jan. 14; 24(2):179-194, Roth, et al., Sci Rep. 2019 Jun. 21; 9(1):9046, and Boland, et al., World J Gastroenterol. 2019 Sep. 7; 25(33):4904-4920, which are hereby incorporated by reference in their entirety.

In this study, ob/ob-NASH mice are divided into 4 ob/ob-NASH groups (e.g. n=14 for each group) with dosing for 12 weeks (PO, QD). Male B6.V-Lep^{ob}/JRj mice are fed 40% HFD, 20% fructose, 2% Cholesterol (GAN) diet for 12+ weeks prior to study start.

All mice entering the experiment are pre-biopsied at week −4 and stratified based on liver biopsy with only animals with fibrosis stage ≥1, inflammation score ≥2 and steatosis score ≥2 being included in the study. Animals are randomized into groups based on fibrosis stage as measured by picosirius red (PSR) staining. Total of 12 weeks of PO, QD dosing. The four groups are as follows: 1) Vehicle, 2) CHS-131, 30 mg/kg, 3) Vitamin E, 2 mg/kg, 4) CHS-131, 30 mg/kg+Vitamin E, 2 mg/kg.

Body weight is measured daily during the study period. Four hour fasting plasma glucose and HbA1c are measured at baseline, week 6, and week 12. Fasting plasma insulin and terminal plasma ALT/AST/GGT/and lipids are also measured at baseline and at week 12.

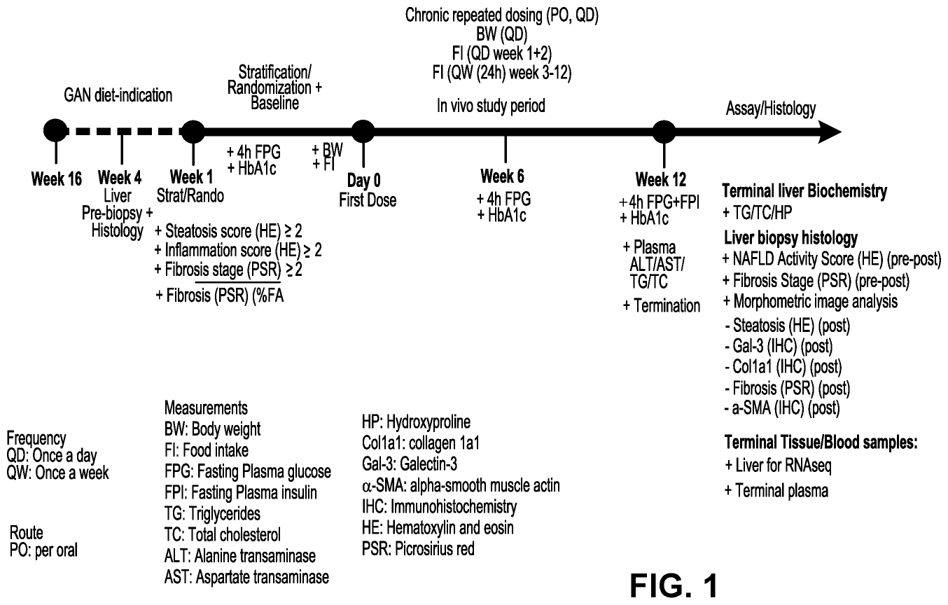

Terminal liver removal, weighing, and sampling at week 12 includes 1) FFPE (histology), 2) biochemical analysis, and 3) RNAseq analysis. Liver biopsy histology includes determination of 1) pre-to-post NAFLD Activity Score including Fibrosis Stage, 2) post steatosis (HE), 3) post Galectin-3 (IHC), an inflammation biomarker; other marker of an inflammatory response such as eicosanoids, hydroxyeicosatetraenoic acids (HETEs) and prostaglandins, are also measured, 4) post-fibrosis (PSR), 5) fibrosis biomarkers, including post Col1a1 (IHC), 6) post a-SMA (IHC). Additional fibrosis biomarkers are optionally measured including Pro-C3, C3M, Pro-C6 and C6M (Nordic Biosciences, Herlev, Denmark) which may characterize an observed anti-fibrotic effect. Liver TG/TC/HP content is also determined. Total adiponectin is measured at baseline and end-of-study. A study outline is shown in FIG. 1.

1-77. (canceled)

78. A method of treating non-alcoholic fatty liver disease (NAFLD) in a subject in need thereof comprising administering to the subject
(a) the compound of Formula (I), 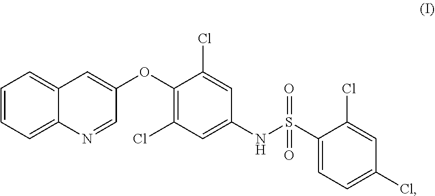 [SMILES: O=S(=O)(Nc1cc(Cl)c(Oc2cnc3ccccc3c2)c(Cl)c1)c1ccc(Cl)cc1Cl]
or a pharmaceutically acceptable salt thereof, and
(b) an antioxidant, or a pharmaceutically acceptable salt thereof,
wherein the amounts of (a) and (b) together are effective in treating NAFLD.

79. The method claim 78, wherein the antioxidant, or a pharmaceutically acceptable salt thereof, is a vitamin or mineral, or a pharmaceutically acceptable salt thereof.

80. The method claim 78, wherein the NAFLD activity score (NAS) following administration is 7 or less.

81. The method claim 80, wherein the NAS is 5 or less.

82. The method claim 80, wherein the NAS is 3 or less.

83. The method claim 78, wherein the NAFLD is simple nonalcoholic fatty liver (NAFL).

84. The method claim 78, wherein the NAFLD is nonalcoholic steatohepatitis (NASH).

85. The method claim 84, wherein the NAFLD is NASH with attendant liver cirrhosis.

86. The method claim 84, wherein the treatment of NASH decreases the level of serum bile acids in the subject.

87. The method claim 84, wherein the treatment of NASH comprises treatment of pruritus.

88. The method claim 78, wherein the antioxidant is selected from the group consisting of: a vitamin, a mineral, a polyphenol, an anthocyanin, a carotenoid, or a glucosinolate, or a pharmaceutically acceptable salt, prodrug, isomer, or a combination of two or more of any of the foregoing.

89. The method claim 88, wherein the antioxidant, or a pharmaceutically acceptable salt thereof, is administered at a dose from about 10 to about 100 mg; or from about 100 to about 250 mg; or from about 250 to about 500 mg; or from about 500 mg to about 1 g; or from about 1 g to about 1.5 g.

90. The method claim 78, wherein the compound of Formula (I), or a pharmaceutically acceptable salt thereof, is administered at a dose from about 0.1 to about 15 mg.

91. The method claim 78, wherein the compound of Formula (I) is in the form of a besylate salt.

92. A method of treating fibrosis in a subject in need thereof comprising administering to the subject
(a) the compound of Formula (I), 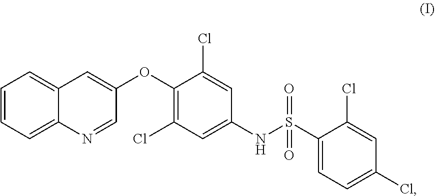 [SMILES: O=S(=O)(Nc1cc(Cl)c(Oc2cnc3ccccc3c2)c(Cl)c1)c1ccc(Cl)cc1Cl]
or a pharmaceutically acceptable salt thereof, and
(b) an antioxidant, or a pharmaceutically acceptable salt thereof,
wherein the amounts of (a) and (b) together are effective in treating fibrosis.

93. The method claim 92, wherein the treatment of fibrosis comprises a decrease in the stage of fibrosis; wherein the decrease in the stage of fibrosis is from stage 4 to stage 3, from stage 4 to stage 2, from stage 4 to stage 1, from stage 4 to stage 0, from stage 3 to stage 2, from stage 3 to stage 1, from stage 3 to stage 0, from stage 2 to stage 1, from stage 2 to stage 0, or from stage 1 to stage 0.

94. The method claim 92, wherein the antioxidant is selected from the group consisting of: a vitamin, a mineral, a polyphenol, an anthocyanin, a carotenoid, or a glucosinolate, or a pharmaceutically acceptable salt, prodrug, isomer, or a combination of two or more of any of the foregoing.

95. The method claim 94, wherein the antioxidant, or a pharmaceutically acceptable salt thereof, is administered at a dose from about 10 to about 100 mg; or from about 100 to about 250 mg; or from about 250 to about 500 mg; or from about 500 mg to about 1 g; or from about 1 g to about 1.5 g.

96. The method claim 92, wherein the compound of Formula (I), or a pharmaceutically acceptable salt thereof, is administered at a dose from about 0.1 to about 15 mg.

97. The method claim 92, wherein the compound of Formula (I) is in the form of a besylate salt.
